(19) United States
(12) Patent Application Publication
(10) Pub. No.: US 2013/0183268 A1
(43) Pub. Date: Jul. 18, 2013

(54) DRUG COMBINATIONS WITH FLUORO-SUBSTITUTED OMEGA-CARBOXYARYL DIPHENYL UREA FOR THE TREATMENT AND PREVENTION OF DISEASES AND CONDITIONS

(71) Applicants: Olaf Christensen, Berlin (DE); Iris Kuss, Berlin (DE)

(72) Inventors: Olaf Christensen, Berlin (DE); Iris Kuss, Berlin (DE)

(73) Assignee: BAYER HEALTHCARE LLC, TARRYTOWN, NY (US)

(21) Appl. No.: 13/810,972

(22) PCT Filed: Jul. 19, 2011
(86) PCT No.: PCT/US11/44506

§ 371 Date: Mar. 29, 2013

Related U.S. Application Data
(60) Provisional application No. 61/365,547, filed on Jul. 19, 2010.

(51) Int. Cl. A61K 31/44; A61K 31/519; A61K 45/06; A61K 33/24

(52) U.S. Cl. CPC A61K 31/44; A61K 33/24; A61K 31/519; A61K 45/06
USPC 424/85.4; 514/350; 424/649; 514/265.1; 424/94.6; 424/133.1

(57) ABSTRACT

The present invention relates to drug combinations and pharmaceutical compositions for treating hyperproliferative disorders such as cancer including non-small cell lung carcinoma, said drug combination comprising (1) a fluoro-substituted-diaryl urea of Formula (I), (2) at least one antifolate and optionally (3) at least one platinum complex antineoplastic nucleic acid binding agent, where any of these components can be present in the form of a pharmaceutically acceptable salt or other derivative thereof.

This application claims priority to U.S. provisional application 61/365,547, filed Jul. 19, 2010, which is incorporated by reference in its entirety.

### FIELD OF THE INVENTION

This invention relates to drug combinations of fluoro substituted omega-carboxyaryl diphenyl ureas with folate antimetabolite chemotherapeutic agents and their use in treating and preventing diseases and conditions, including hyper-proliferative disorders such as cancer in humans and other mammals.

### BACKGROUND OF THE INVENTION

Substituted diarylureas are a class of serine-threonine kinase inhibitors as well as tyrosine kinase inhibitors known in the art (Smith et al., Bioorg. Med. Chem. Lett. 2001, 11, 2775-2778, Lowinger et al., Clin. Cancer Res. 2000, 6(suppl.), 335, Lyons et al., Endocr.-Relat. Cancer 2001, 8, 219-225, Lowinger et al., Curr. Pharm. Design 2002, 8, 99-110). Omega-carboxyaryl diphenyl ureas are disclosed in WO00/42012 and WO00/41698 and fluoro-substituted omega-carboxyaryl diphenyl ureas are disclosed in WO/2005/009961. In particular, it is disclosed that the fluoro-substituted diphenyl urea of formula (I)

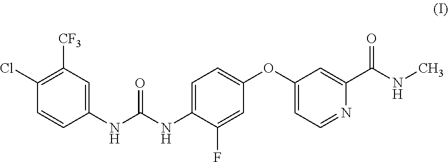 [SMILES: CNC(=O)c1cc(Oc2ccc(NC(=O)Nc3ccc(Cl)c(C(F)(F)F)c3)c(F)c2)ccn1]

also referred as “regorafenib” or “4{4-[3-(4-chloro-3-trifluoromethylphenyl)-ureido]-3-fluorophenoxy}-pyridine-2-carboxylic acid methylamide” or “N-(4-chloro-3-(trifluoromethyl)phenyl-N′-(4-(2-(N-methylcarbamoyl)-4-pyridyloxy)-2-fluorophenyl)urea,” and polymorphs, solvates, hydrates, metabolites, prodrugs, pharmaceutically acceptable salts or diastereoisomers thereof, are potent inhibitors of raf, VEGFR, p38, PDGFR and/or flt-3 signaling kinases. These enzymes are all molecular targets of interest for the treatment of hyper-proliferative diseases, including cancer.

WO 03/047579 relates to the use of substituted diaryl ureas in combination with cytotoxic or cytostatic compounds for treating cancer.

Methods for preparing the fluoro-substituted diaryl ureas of Formula (I) and polymorphs, solvates, hydrates, metabolites, prodrugs, pharmaceutically acceptable salts or diastereoisomers thereof are described in the following applications:

WO/2005/009961, filed Jul. 22, 2004,

WO/2006/026500, filed Aug. 29, 2005,

WO/2008/043446, filed Sep. 29, 2007,

WO/2008/089389, filed Jan. 18, 2008,

WO/2008/089388, filed Jan. 18, 2008,

US 2008-0262236, filed May 21, 2008,

US 2008-02427607, filed Jun. 9, 2008,

US 2009-0306020, filed Apr. 22, 2009,

US 2010/0063112, filed Mar. 11, 2010, and

US 2010/0113533, filed May 6, 2010.

The compound of the formula (I) prepared in the manner described in WO 2005/009961 corresponds to polymorph I having a melting point of 186-206° C. A characteristic X-ray diffractogram, IR spectrum, Raman spectrum, FIR spectrum, NIR spectrum and a ¹³C-solid state-NMR spectrum for polymorph I is shown in FIGS. 2-7 in each of Published US Application Nos. 2010/0113533 and 2010/0063112. The present invention includes the polymorph II (which melts at 181° C.) and polymorph III (which melts at 141 C.°) of 4-[4-({[4-chloro-3-(trifluoromethyl)phenyl]carbamoyl}amino)-3-fluorophenoxy]-N-methylpyridine-2-carboxamide, which are disclosed in Published US Application Nos. 2010/0113533 and 2010/0063112, respectively.

In comparison to the polymorph I of the compound of the formula (I), polymorphs II and III have a clearly differentiable X-ray diffractogram, IR spectrum, Raman spectrum, FIR spectrum, NIR spectrum and ¹³C-solid state NMR spectrum as shown in FIGS. 2-7 of Published US Application Nos. 2010/0113533 and 2010/0063112, respectively.

A class of antimetobolite chemotherapeutic drugs known as folate antimetabolites, folate antagonists and antifolates act by inhibiting the metabolism of folic acid. These will be referred to herein as antifolates. Antifolates interfere with cell metabolic processes that are dependent on folate and are required for cell replication. When these substances are incorporated into the cellular metabolism, they produce an intracellular state of folic acid deficiency in order to inhibit folate-dependant enzymes along the folate metabolic pathway. DNA synthesis and cell division, processes involved in malignant tumor growth, are hindered by this folic acid deficiency. Patients treated with antifolates typically take vitamin B12 and folic acid supplements to help control the hematologic and GI toxicities of the antifolates.

Currently the forms of cancer which are being treated with antifolate chemotherapy include: breast cancer, head and neck cancer, bladder cancer, acute lymphocytic leukemia, non-Hodgkin's lymphoma, choriocarcinoma, and osteogenic sarcoma. Antifolates are also being used in the treatment of non-cancerous diseases such as malaria, bacterial infections, psoriasis, and rheumatoid arthritis.

Methotrexate (formerly known as amethopterin), is an antifolate and is one of the early chemotherapy drugs having been developed in the late 1940s. Since then, a series of 4-hydroxypyrrolo[2,3-d]pyrimidine-L-glutamic acid derivatives of the formula X below and salts thereof with antifolate activity have been disclosed and shown to be particularly useful antifolate drugs. See, e.g., Akimoto, et al., U.S. Pat. Nos. 4,997,838, 5,106,974 and 5,539,113.

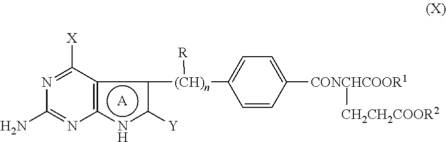

wherein the ring A is a pyrrole or pyrroline ring, X is an amino group or a hydroxyl group, Y is a hydrogen atom, an amino group or a hydroxyl group, R is a hydrogen atom, a fluorine atom, a C₁₋₆ alkyl group, an alkenyl group or an alkynyl group, R¹ and —R² are independently a hydrogen atom or C₁₋₆ alkyl and n is an integer of 2 to 4, and R may be different in each of the n repeating units.

The art recognizes multiple methods for the preparation of glutamic acid derivatives, some of which are disclosed in U.S. Pat. Nos. 4,997,838, 5,106,974, 5,416,211 and 5,539,113.

Glutamic acid derivatives of particular interest are of the formula XX below which are disclosed in U.S. Pat. Nos. 4,996,206 and 5,344,932.

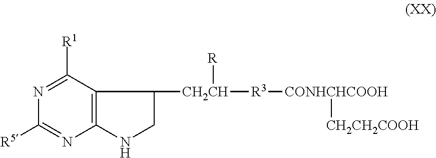

wherein R¹ is —OH or —NH₂,
R³ is 1,4-phenylene or 1,3-phenylene unsubstituted or substituted with chloro, fluoro, methyl, methoxy, or trifluoromethyl; thienediyl or furanediyl each unsubstituted or substituted with chloro, fluoro, methyl, methoxy, or trifluoromethyl; cyclohexanediyl; or alkanediyl;
R is hydrogen, methyl, or hydroxymethyl;
R^{5′} is hydrogen or alkyl of 1 to 6 carbon atoms; and
the pharmaceutically acceptable salts thereof.

A folate antagonist of particular interest is the glutamic acid derivative Pemetrexed, (S)-2-[4-[2-(4-amino-2-oxo-3,5,7-triazabicyclo[4.3.0]nona-3,8,10-trien-9-yl)ethyl]benzoyl]aminopentanedioic acid, of the formula (A) below and pharmaceutically acceptable salts thereof.

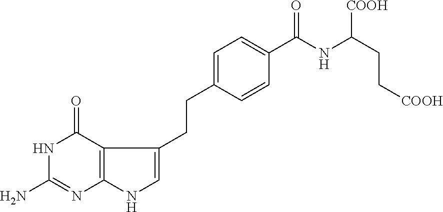 [SMILES: Nc1nc2[nH]cc(CCc3ccc(C(=O)NC(CCC(=O)O)C(=O)O)cc3)c2c(=O)[nH]1]

Methods suitable for preparing Pemetrexed, are described in U.S. Pat. Nos. 4,996,206, 5,344,932 and 5,416,211.

Pemetrexed, also known by the brand name Alimta®, was developed and is now manufactured and marketed by Eli Lilly and Company, an Indianapolis based company. It is reported the Premetrexed inhibits a number of enzymes that are required for purine and pyrimidine synthesis, which prevents the formation of DNA and RNA required for growth and survival of both cancer and normal cells. These enzymes include thymidylate synthase (TS), dihydrofolate reductase (DHFR), and glycinamide ribonucleotide formyl transferase (GARFT).

Pemetrexed was approved by the United States Food and Drug administration in February 2004 for the treatment of malignant pleural mesothelioma (MPM), a type of tumor of the lining of the lung, in combination with cisplatin, a platinum-containing chemotherapeutic drug. In July 2004, the drug was approved by the FDA as a second line agent for the treatment of advanced or metastatic non-small cell lung cancer (NSCLC). In September 2008, the FDA granted approval as a first-line treatment, in combination with cisplatin, against locally-advanced and metastatic NSCLC, in patients with non-squamous histology. Other known antifolates which are not a pyrrolopyrimidine-L-glutamic acid derivatives are Trimethoprim(5-(3,4,5-trimethoxybenzyl)pyrimidine-2,4-diamine) and pyrimethamine(5-(4-chlorophenyl)-6-ethyl-2,4-pyrimidinediamine).

The use of platinum coordination complexes as chemotherapy drugs to treat various types of cancers, including sarcomas, some carcinomas (e.g. small cell lung cancer, and ovarian cancer), lymphomas, and germ cell tumors is well known. The cytotoxicity of platinum compounds is thought to result from inhibition of DNA synthesis in cancer cells by binding to the nucleic acid and ultimately triggering apoptosis.

Common platinum coordination complexes are Cisplatin, (cis-diamminedichloroplatinum(II)); Carboplatin, (cis-diammine(cyclobutane-1,1-dicarboxylate-O,O′)platinum(II); Oxaliplatin, ([(1R,2R)-cyclohexane-1,2-diamine](ethanedioato-O,O′)platinum(II)); Tetraplatin or Ormaplatin ((1R,2R)-cyclohexane-1,2-diamine platinum(IV) tetrachloride) and Satraplatin, ((OC-6-43)-bis(acetato)aminedichloro(cyclohexylamine)platinum).

Cisplatin-containing and carboplatin-containing combination chemotherapy regimens are reported to produce objective response rates (including a few complete responses) that are higher than those achieved with single-agent chemotherapy. Although toxic effects may vary, outcome is similar with most cisplatin-containing regimens; a randomized trial comparing 5 cisplatin-containing regimens reported no significant difference in response, duration of response, or survival.[1] Patients with good performance status and a limited number of sites of distant metastases were reported to have superior response and survival when given chemotherapy when compared to other patients.[2].

These results support further evaluation of chemotherapeutic approaches for both metastatic and locally advanced non-small cell lung cancer (NSCLC). No specific regimen has been regarded as standard therapy. Radiation therapy may be effective in palliating symptomatic local involvement with NSCLC such as tracheal, esophageal, or bronchial compression, bone or brain metastases, pain, vocal cord paralysis, hemoptysis, or superior vena cava syndrome.

The following Chemotherapy regimens have been associated with similar survival outcomes:
cisplatin plus vinblastine plus mitomycin [15]
cisplatin plus vinorelbine [3]
cisplatin plus paclitaxel [8]
cisplatin plus gemcitabine [16]
carboplatin plus paclitaxel [6,7]

### REFERENCES

1. Weick J K, Crowley J, Natale R B, et al.: A randomized trial of five cisplatin-containing treatments in patients with metastatic non-small-cell lung cancer: a Southwest Oncology Group study. Journal of Clinical Oncology 9(7): 1157-1162, 1991.
2. O'Connell J P, Kris M G, Gralla R J, et al.: Frequency and prognostic importance of pretreatment clinical characteristics in patients with advanced non-small-cell lung cancer treated with combination chemotherapy. Journal of Clinical Oncology 4(11): 1604-1614, 1986.
3. Le Chevalier T, Brisgand D, Douillard J Y, et al.: Randomized study of vinorelbine and cisplatin versus vindesine and cisplatin versus vinorelbine alone in advanced non-small-cell lung cancer: results of a European multicenter trial including 612 patients. Journal of Clinical Oncology 12(2): 360-367, 1994.
4. Chang A Y, Kim K, Glick J, et al.: Phase II study of taxol, merbarone, and piroxantrone in stage IV non-small-cell lung cancer: the Eastern Cooperative Oncology Group results. Journal of the National Cancer Institute 85(5): 388-394, 1993.
5. Murphy W K, Fossella F V, Winn R J, et al.: Phase II study of taxol in patients with untreated advanced non-small-cell lung cancer. Journal of the National Cancer Institute 85(5): 384-388, 1993.
6. Johnson D H, Paul D M, Hande K R, et al.: Paclitaxel plus carboplatin in advanced non-small-cell lung cancer: a phase II trial. Journal of Clinical Oncology 14(7): 2054-2060, 1996.
7. Langer C J, Leighton J C, Comis R L, et al.: Paclitaxel and carboplatin in combination in the treatment of advanced non-small-cell lung cancer: a phase II toxicity, response, and survival analysis. Journal of Clinical Oncology 13(8): 1860-1870, 1995.
8. Bonomi P, Kim K, Chang A, et al.: Phase III trial comparing etoposide (E) cisplatin (C) versus taxol (T) with cisplatin-G-CSF(G) versus taxol-cisplatin in advanced non-small cell lung cancer. An Eastern Cooperative Oncology Group (ECOG) trial. Proceedings of the American Society of Clinical Oncology 15: A-1145, 382, 1996.
9. Souquet P J, Chauvin F, Boissel J P, et al.: Polychemotherapy in advanced non small cell lung cancer: a meta-analysis. Lancet 342(8862): 19-21, 1993.

Non-small cell lung cancer (NSCLC) is a heterogeneous aggregate of at least three different histologies of lung cancer including epidermoid or squamous carcinoma, adenocarcinoma, and large cell carcinoma. They are often classified together because, in their localized states, all have the potential for cure with surgical procedure. At diagnosis, patients with NSCLC can be divided into three groups that reflect the extent of disease and treatment approach. The first group is characterized by surgically resectable tumors, and can be staged I or II. This is the group with the best prognosis, depending on a variety of tumor and host factors. The second group includes patients with advanced lung cancer and can be sub-categorized as local or regional. Radiation therapy with or without chemotherapy or other therapy modalities is the preferred mode of treatment. The final group comprises patients with distant metastasis. This group can be treated with radiation therapy or chemotherapy for palliation of symptoms from the primary tumor. Cisplatin-based chemotherapy has been associated with short-term palliation of symptoms and a small survival advantage.

For operable patients, prognosis is adversely influenced by the presence of pulmonary symptoms, large tumor size (>3 centimeters), and presence of the Erb-2 oncoprotein.[1-6] Other factors that have been identified as adverse prognostic factors in some series of patients with resectable non-small cell lung cancer include mutation of the K-ras gene, vascular invasion, and increased numbers of blood vessels in the tumor specimen.[3,7,8]

### REFERENCES

1. Albain K S, Crowley J J, LeBlanc M, et al.: Survival determinants in extensive-stage non-small-cell lung cancer: the Southwest Oncology Group experience. Journal of Clinical Oncology 9(9): 1618-1626, 1991.
2. Macchiarini P, Fontanini G, Hardin M J, et al.: Blood vessel invasion by tumor cells predicts recurrence in completely resected T1 N0 M0 non-small-cell lung cancer. Journal of Thoracic and Cardiovascular Surgery 106(1): 80-89, 1993.
3. Harpole D H, Herndon J E, Wolfe W G, et al.: A prognostic model of recurrence and death in stage I non-small cell lung cancer utilizing presentation, histopathology, and oncoprotein expression. Cancer Research 55(1): 51-56, 1995.
4. Ichinose Y, Yano T, Asoh H, et al.: Prognostic factors obtained by a pathologic examination in completely resected non-small-cell lung cancer: an analysis in each pathologic stage. Journal of Thoracic and Cardiovascular Surgery 110(3): 601-605, 1995.
5. Martini N, Bains M S, Burt M E, et al.: Incidence of local recurrence and second primary tumors in resected stage I lung cancer. Journal of Thoracic and Cardiovascular Surgery 109(1): 120-129, 1995.
6. Strauss G M, Kwiatkowski D J, Harpole D H, et al.: Molecular and pathologic markers in stage I non-small-cell carcinoma of the lung. Journal of Clinical Oncology 13(5): 1265-1279, 1995.
7. Slebos R J, Kibbelaar R E, Dalesio O, et al.: K-RAS oncogene activation as a prognostic marker in adenocarcinoma of the lung. New England Journal of Medicine 323(9): 561-565, 1990.
8. Fontanini G, Bigini D, Vignati S, et al.: Microvessel count predicts metastatic disease and survival in non-small cell lung cancer. Journal of Pathology 177: 57-63, 1995.

Prior to initiating treatment of any patient with lung cancer, a review of pathologic material by an experienced lung cancer pathologist can be important since the chemo-responsive small cell lung cancer can be confused with non-small cell carcinoma [1]. Histologic classification of non-small cell lung cancer can be squamous cell (epidermoid) carcinoma, adenocarcinoma, large cell carcinoma, adenosquamous carcinoma, and undifferentiated carcinoma. Similarly the staging procedure can be performed using the guidelines set by the American Joint Committee on Cancer (AJCC). Since the classification is based on characterization of the primary tumor (T), measurement of the size of lymph node (N), and assessment of distant metastasis (M), it is shortly known as TNM classification system for NSCLC.

In advanced-stage disease, chemotherapy has been reported to offer modest improvements in median survival.[1,2] Chemotherapy has been reported to produce short-term improvement in disease-related symptoms, while combination chemotherapy has been reported to yield symptomatic relief [3,4].

### REFERENCES

1. Souquet P J, Chauvin F, Boissel J P, et al.: Polychemotherapy in advanced non small cell lung cancer: a meta-analysis. Lancet 342(8862): 19-21, 1993.
2. Non-small Cell Lung Cancer Collaborative Group: Chemotherapy in non-small cell lung cancer: a meta-analysis using updated data on individual patients from 52 randomised clinical trials. British Medical Journal 311(7010): 899-909, 1995.
3. Hardy J R, Noble T, Smith I E: Symptom relief with moderate dose chemotherapy (mitomycin-C, vinblastine and cisplatin) in advanced non-small cell lung cancer. British Journal of Cancer 60(5): 764-766, 1989.
4. Ellis P A, Smith I E, Hardy J R, et al.: Symptom relief with MVP (mitomycin C, vinblastine and cisplatin) chemotherapy in advanced non-small-cell lung cancer. British Journal of Cancer 71(2): 366-370, 1995.

The lung is also frequently the site of second primary malignancies in patients with primary lung cancers. Determining whether the new lesion is a new primary cancer or a metastasis may be difficult. Studies have indicated that in the majority of patients the new lesion is a second primary tumor, and following resection some patients may achieve long-term survival. Thus, if the first primary tumor has been controlled, the second primary tumor should be resected if possible.[8,9] It is reported that the use of chemotherapy has produced objective responses and small improvement in survival for patients with metastatic disease.[10]

### Treatment Options:

1. Palliative radiation therapy.
2. Chemotherapy alone. For patients who have not received prior chemotherapy, the following regimens are associated with similar survival outcomes:
   cisplatin plus vinblastine plus mitomycin [13]
   cisplatin plus vinorelbine [14]
   cisplatin plus paclitaxel [15]
   cisplatin plus gemcitabine [16]
   carboplatin plus paclitaxel [17,18]
   3. Surgical resection of isolated cerebral metastasis (highly selected patients).[6]
   4. Laser therapy or interstitial radiation therapy for endobronchial lesions.[19]
   5. Stereotactic radiosurgery (highly selected patients).[3,5]

### REFERENCES

1. Patchell R A, Tibbs P A, Walsh J W, et al.: A randomized trial of surgery in the treatment of single metastases to the brain. New England Journal of Medicine 322(8): 494-500, 1990.
2. Mandell L, Hilaris B, Sullivan M, et al.: The treatment of single brain metastasis from non-oat cell lung carcinoma: surgery and radiation versus radiation therapy alone. Cancer 58(3): 641-649, 1986.
3. Loeffler J S, Kooy H M, Wen P Y, et al.: The treatment of recurrent brain metastases with stereotactic radiosurgery. Journal of Clinical Oncology 8(4): 576-582, 1990.
4. DeAngelis L M, Mandell L R, Thaler H T, et al.: The role of postoperative radiotherapy after resection of single brain metastases. Neurosurgery 24(6): 798-805, 1989.
5. Alexander E, Moriarty T M, Davis R B, et al.: Stereotactic radiosurgery for the definitive, noninvasive treatment of brain metastases. Journal of the National Cancer Institute 87(1): 34-40, 1995.
6. Arbit E, Wronski M, Burt M, et al.: The treatment of patients with recurrent brain metastases: a retrospective analysis of 109 patients with nonsmall cell lung cancer. Cancer 76(5): 765-773, 1995.
7. Hazuka M B, Kinzie J J: Brain metastases: results and effects of re-irradiation. International Journal of Radiation Oncology, Biology, Physics 15(2): 433-437, 1988.
8. Salerno T A, Munro D D, Blundell P E, et al.: Second primary bronchogenic carcinoma: life-table analysis of surgical treatment. Annals of Thoracic Surgery 27(1): 3-6, 1979.
9. Yellin A, Hill L R, Benfield J R: Bronchogenic carcinoma associated with upper aerodigestive cancer. Journal of Thoracic and Cardiovascular Surgery 91(5): 674-683, 1986.
10. Souquet P J, Chauvin F, Boissel J P, et al.: Polychemotherapy in advanced non small cell lung cancer: a meta-analysis. Lancet 342(8862): 19-21, 1993.
11. Ellis P A, Smith I E, Hardy J R, et al.: Symptom relief with MVP (mitomycin C, vinblastine and cisplatin) chemotherapy in advanced non-small-cell lung cancer. British Journal of Cancer 71(2): 366-370, 1995.
12. Medical Research Council Lung Cancer Working Party: Randomized trial of etoposide cyclophosphamide methotrexate and vincristine versus etoposide and vincristine in the palliative treatment of patients with small-cell lung cancer and poor prognosis. British Journal of Cancer 67(Suppl 20): A-4;2, 14, 1993.
13. Veeder M H, Jett J R, Su J Q, et al.: A phase III trial of mitomycin C alone versus mitomycin C, vinblastine, and cisplatin for metastatic squamous cell lung carcinoma. Cancer 70(9): 2281-2287, 1992.
14. Le Chevalier T, Brisgand D, Douillard J Y, et al.: Randomized study of vinorelbine and cisplatin versus vindesine and cisplatin versus vinorelbine alone in advanced non-small-cell lung cancer: results of a European multicenter trial including 612 patients. Journal of Clinical Oncology 12(2): 360-367, 1994.
15. Bonomi P, Kim K, Chang A, et al.: Phase III trial comparing etoposide (E) cisplatin (C) versus taxol (T) with cisplatin-G-CSF(G) versus taxol-cisplatin in advanced non-small cell lung cancer. An Eastern Cooperative Oncology Group (ECOG) trial. Proceedings of the American Society of Clinical Oncology 15: A-1145, 382, 1996.
16. Rosell R, Tonato M, Sandler A: The activity of gemcitabine plus cisplatin in randomized trials in untreated patients with advanced non-small cell lung cancer. Seminars in Oncology 25(4 suppl 9): 27-34, 1998.
17. Johnson D H, Paul D M, Hande K R, et al.: Paclitaxel plus carboplatin in advanced non-small-cell lung cancer: a phase II trial. Journal of Clinical Oncology 14(7): 2054-2060, 1996.
18. Langer C J, Leighton J C, Comis R L, et al.: Paclitaxel and carboplatin in combination in the treatment of advanced non-small-cell lung cancer: a phase II toxicity, response, and survival analysis. Journal of Clinical Oncology 13(8): 1860-1870, 1995.
19. Miller J I, Phillips T W: Neodymium:YAG laser and brachytherapy in the management of inoperable bronchogenic carcinoma. Annals of Thoracic Surgery 50(2): 190-196, 1990.

### DESCRIPTION OF THE INVENTION

The present invention provides drug combinations, pharmaceutical compositions, and methods for treating diseases and conditions, including, but not limited to, cell proliferative disorders such as cancer, including but not limited to colon, gastric, lung (NSCLC), pancreatic, thyroid, ovarian, prostate, leukemia, melanoma, hepatocellular, renal, head and neck, glioma and mammary cancers and gastrointestinal stromal tumors.

The drug combinations comprise (1) at least one fluoro-substituted-diaryl urea of Formula I (defined above) and (2) at least one antifolate such as Pemetrexed (Alimta®), and optionally (3) at least one platinum complex antineoplastic nucleic acid binding agent such as carboplatin (Paraplatin®), oxaplatin (Eloxatin®), cisplatin (Platinol®), Tetraplatin or Ormaplatin, and Satraplatin (Spera™), where any of these components can be present in the form of a pharmaceutically acceptable salt or other known derivative. The drug combinations of the invention can be formed in vivo, e.g., in a patient's body.

In a preferred embodiment, the drug combination is the fluoro-substituted diaryl urea of formula I, Pemetrexed (Alimta®) and cisplatin.

The invention also relates to pharmaceutical compositions which comprise one or more pharmaceutically acceptable carrier molecules and quantities of fluoro-substituted diaryl urea compound of Formula I (defined above), an antifolate (e.g., Pemetrexed (Alimta®) and optionally a platinum complex (e.g., cisplatin), in amounts which are jointly effective for treating a cancer, where any of these components can be present in the form of a pharmaceutically acceptable salt or other common derivative.

The methods of this invention include, for example, administering (1) a fluoro-substituted diaryl urea compound of Formula I (e.g. Regorafenib); (2) an antifolate (e.g. Pemetrexed (Alimta®) and optionally (3) a platinum complex (e.g., cisplatin) or pharmaceutically-acceptable salts or derivatives thereof, etc.

In particular embodiments of this invention, the active components of the drug combination are administered to a patient by oral delivery and/or by intravenous injection or infusion.

In particular embodiments of this invention, the fluoro-substituted diaryl urea compound of Formula I is administered simultaneously with the antifolate (e.g. Pemetrexed (Alimta®) and optionally with (3) a platinum complex (e.g., cisplatin) to a patient with cancer, in the same formulation or in separate formulations, optionally using different administration routes. Administration can also be sequentially, in any order.

In particular embodiments of this invention, the fluoro-substituted diaryl urea compounds of Formula I (e.g., Regorafenib) are administered in tandem with the antifolate (e.g. Pemetrexed (Alimta®)) and optionally with a platinum complex (e.g., cisplatin), wherein the fluoro-substituted diaryl urea compound of Formula I is administered to a patient once or more per day for up to 28 consecutive days with the concurrent or intermittent administration of the antifolate and optional platinum complex over the same total time period.

In particular embodiments of this invention, the fluoro-substituted diaryl urea compound of Formula I (e.g., Regorafenib) can be administered to a patient as an oral, intravenous, intramuscular, subcutaneous, or parenteral dosage which can range from about 0.1 to about 300 mg/kg of total body weight.

In particular embodiments of this invention, the antifolate is administered at a conventional dosage level, at a conventional dosage rate by a conventional method of administration. Pemetrexed is typically administered as a solution (500 mg/m², 500 mg for every square meter (m²) of the patient's surface area) via injection into a vein (10-minute infusion) once every 21 days.

In particular embodiments of this invention, the optional platinum complex is administered at a conventional dosage level, at a conventional dosage rate by conventional means. Cisplatin is typically administered intravenously as a sterile aqueous solution. A single dose intended for a 3-4 week period can range from 50 to 100 mg/m² (patient surface area). A daily dose of 15 to 20 mg/m² for 5 days every 3 to 4 weeks is an alternative to a single dose.

In particular embodiments of this invention, the fluoro-substituted diaryl urea compound of Formula I is administered in solid dispersion, the synthesis of which is disclosed in WO/2006/026500, filed Aug. 29, 2005, with examples which are incorporated herein by reference.

This invention also relates to combinations, pharmaceutical compositions methods comprising a substituted diaryl urea compound of formula I, an antifolate and a platinum complex in amounts adjusted for the concurrent use of these agents.

This invention further relates to kits where the dosages of the three chemotherapeutic agents are in at least two separate containers.

The present invention also relates to useful forms of the fluoro-substituted aryl urea of Formula (I) and the antifolates (e.g. Pemetrexed), and platinum complexes. These include polymorphs, solvates, hydrates, metabolites, prodrugs, pharmaceutically acceptable salts or diastereoisomers of the fluoro-substituted aryl urea of Formula (I), antifolates and platinum complexes.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aryl or aralkyl halides like benzyl and phenethyl bromides and others monosubstituted aralkyl halides or polysubstituted aralkyl halides.

Solvates of the fluoro-substituted diaryl urea compound of Formula I, antifolate (e.g. Pemetrexed (Alimta®), and optional a platinum complex (e.g., cisplatin) for the purposes of the invention are those forms of the compounds where solvent molecules form a complex in the solid state and include, but are not limited to for example ethanol and methanol.

Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the fluoro-substituted diaryl urea compound of Formula I, antifolates and optional platinum complex used in this invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: New York (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff, M. E. ed. Burgers Medicinal Chemistry And Drug Discovery John Wiley & Sons: New York (1997), 949-982.

Active metabolites of the fluoro-substituted aryl urea of Formula (I) and antifolates are included in his invention. The metabolites of the fluoro-substituted aryl urea of Formula (I) include oxidized derivatives where the metabolism site is either one of the two urea nitrogen atoms, or the pyridine nitrogen atom, or the methylamide functionality, or any combination thereof. Oxidation typically results in either urea nitrogen atom carrying a hydroxyl group, and/or the pyridine nitrogen atom being substituted by oxygen (referred to in the art as 1-oxo-pyridine) or hydroxy (referred to in the art as 1-hydroxy-pyridine), and/or the amide functionality being de-methylated. Examples include:

4{4-[3-(4-chloro-3-trifluoromethylphenyl)-ureido]-3-fluorophenoxy}-pyridine-2-carboxylic acid amide,

4{4-[3-(4-chloro-3-trifluoromethylphenyl)-ureido]-3-fluorophenoxy}-1-hydroxy-pyridine-2-carboxylic acid methylamide, and

4{4-[3-(4-chloro-3-trifluoromethylphenyl)-ureido]-3-fluorophenoxy}-1-hydroxy-pyridine-2-carboxylic acid amide.

The salts and prodrugs of the fluoro-substituted diaryl urea of Formula (I) and antifolates may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those with the absolute configuration of the compound of Formula (I) which produces the more desirable biological activity. Separated, pure or partially purified isomers or racemic mixtures of derivatives of the compound of Formula (I) are also included within the scope of the present invention. The purification of said isomers and the separation of said isomeric mixtures can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds can likewise be obtained by chiral syntheses utilizing optically active starting materials.

### General Preparative Methods for the Fluoro-Substituted Diaryl Urea of Formula (I)

The fluoro-substituted diaryl urea of Formula (I) may be prepared by use of known chemical reactions and procedures as described in the following published applications

WO 2000/42012, WO 2003/047579, WO 2004/078747, WO 2005/000284, WO2005/009961, WO/2006/026500, WO/2008/043446, WO/2008/089389, WO/2008/089388, US 2008-0262236, US 2008-02427607, US 2009-0192127, and US 2009-0306020.

The fluoro-substituted diaryl urea of Formula (I) can be made according to conventional chemical methods, and/or as disclosed below, from starting materials which are either commercially available or producible according to routine, conventional chemical methods. General methods for the preparation of the fluoro-substituted diaryl urea of Formula (I) are given below and its preparation is specifically illustrated in the examples.

As described in one or more of the published applications above, the fluoro-substituted diaryl urea of Formula (I) can be prepared from the condensation of the two arylamine fragments in the presence of phosgene, di-phosgene, tri-phosgene, carbonyldiimidazole, or equivalents thereof in a solvent that does not react with any of the starting materials, or alternatively, the fluoro-substituted diaryl urea of Formula (I) can be synthesized by reacting amino compounds with isocyanate compounds.

The isocyanates are commercially available or can be synthesized from heterocyclic amines according to methods commonly known to those skilled in the art [e.g. from treatment of an amine with phosgene or a phosgene equivalent such as trichloromethyl chloroformate(diphosgene), bis(trichloromethyl)carbonate (triphosgene), or N,N′-carbonyldiimidazole (CDI); or, alternatively by a Curtius-type rearrangement of an amide, or a carboxylic acid derivative, such as an ester, an acid halide or an anhydride].

Aryl amines are commonly synthesized by reduction of nitroaryls using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroaryls may also be directly reduced using a strong hydride source, such as LiAlH₄ (Seyden-Penne. Reductions by the Alumino- and borohydrides in Organic Synthesis; VCH Publishers: New York (1991)), or using a zero valent metal, such as Fe, Sn or Ca, often in acidic media. Many methods exist for the synthesis of nitroaryls (March. Advanced Organic Chemistry, 3^{rd} Ed.; John Wiley: New York (1985). Larock. Comprehensive Organic Transformations; VCH Publishers: New York (1989)). Nitro aryls are commonly formed by electrophilic aromatic nitration using HNO₃, or an alternative NO₂⁺ source.

Pyridine-1-oxides of Formula (I) where the pyridine ring carries a hydroxy substituent on its nitrogen atom, and A, B, L are broadly defined as above can be prepared from the corresponding pyridines using oxidation conditions known in the art. Some examples are as follows:
peracids such as meta chloroperbenzoic acids in chlorinated solvents such as dichloromethane, dichloroethane, or chloroform (Markgraf et al., Tetrahedron 1991, 47, 183);
(Me₃SiO)₂ in the presence of a catalytic amount of perrhenic acid in chlorinated solvents such as dichloromethane (Coperet et al., Terahedron Lett. 1998, 39, 761);
Perfluoro-cis-2-butyl-3-propyloxaziridine in several combinations of halogenated solvents (Amone et al., Tetrahedron 1998, 54, 7831);
Hypofluoric acid-acetonitrile complex in chloroform (Dayan et al., Synthesis 1999, 1427);
Oxone, in the presence of a base such as KOH, in water (Robker et al., J. Chem. Res., Synop. 1993, 10, 412);
Magnesium monoperoxyphthalate, in the presence of glacial acetic acid (Klemm et al., J. Heterocylic Chem. 1990, 6, 1537);
Hydrogen peroxide, in the presence of water and acetic acid (Lin A. J., Org. Prep. Proced. Int. 1991, 23(1), 114);
Dimethyldioxirane in acetone (Boyd et al., J. Chem. Soc., Perkin Trans. 1991, 9, 2189).

In addition, specific methods for preparing diaryl ureas and intermediate compounds are already described elsewhere in the patent literature, and can be adapted to the compounds of the present invention. For example, Miller S. et al, “Inhibition of p38 Kinase using Symmetrical and Unsymmetrical Diphenyl Ureas” PCT Int. Appl. WO 99 32463, Miller, S et al. “Inhibition of raf Kinase using Symmetrical and Unsymmetrical Substituted Diphenyl Ureas” PCT Int. Appl., WO 99 32436, Dumas, J. et al., “Inhibition of p38 Kinase Activity using Substituted Heterocyclic Ureas” PCT Int. Appl., WO 99 32111, Dumas, J. et al., “Method for the Treatment of Neoplasm by Inhibition of raf Kinase using N-Heteroaryl-N′-(hetero)arylureas” PCT Int. Appl., WO 99 32106, Dumas, J. et al., “Inhibition of p38 Kinase Activity using Aryl- and Heteroaryl-Substituted Heterocyclic Ureas” PCT Int. Appl., WO 99 32110, Dumas, J., et al., “Inhibition of raf Kinase using Aryl- and Heteroaryl-Substituted Heterocyclic Ureas” PCT Int. Appl., WO 99 32455, Riedl, B., et al., “O-Carboxy Aryl Substituted Diphenyl Ureas as raf Kinase Inhibitors” PCT Int. Appl., WO 00 42012, Riedl, B., et al., “O-Carboxy Aryl Substituted Diphenyl Ureas as p38 Kinase Inhibitors” PCT Int. Appl., WO 00 41698, Dumas, J. et al. “Heteroaryl ureas containing nitrogen hetero-atoms as p38 kinase inhibitors” U.S. Pat. Appl. Publ., US 20020065296, Dumas, J. et al. “Preparation of N-aryl-N′-[(acylphenoxy)phenyl]ureas as raf kinase inhibitors” PCT Int. Appl., WO 02 62763, Dumas, J. et al. “Inhibition of raf kinase using quinolyl, isoquinolyl or pyridyl ureas” PCT Int. Appl., WO 02 85857, Dumas, J. et al. “Preparation of quinolyl, isoquinolyl or pyridyl-ureas as inhibitors of raf kinase for the treatment of tumors and/or cancerous cell growth” U.S. Pat. Appl. Publ., US 20020165394. All the preceding patent applications are hereby incorporated by reference.

Synthetic transformations that may be employed in the synthesis of the fluoro-substituted diaryl urea of Formula (I) are known by or accessible to one skilled in the art. Collections of synthetic transformations may be found in compilations, such as:
J. March. Advanced Organic Chemistry, 4^{th} ed.; John Wiley: New York (1992);
R. C. Larock. Comprehensive Organic Transformations, 2^{nd} ed.; Wiley-VCH: New York (1999);
F. A. Carey; R. J. Sundberg. Advanced Organic Chemistry, 2^{nd} ed.; Plenum Press: New York (1984);
T. W. Greene; P. G. M. Wuts. Protective Groups in Organic Synthesis, 3^{rd} ed.; John Wiley: New York (1999);
L. S. Hegedus. Transition Metals in the Synthesis of Complex Organic Molecules, 2^{nd} ed.; University Science Books: Mill Valley, Calif. (1994);
L. A. Paquette, Ed. The Encyclopedia of Reagents for Organic Synthesis; John Wiley: New York (1994);
A. R. Katritzky; O. Meth-Cohn; C. W. Rees, Eds. Comprehensive Organic Functional Group Transformations; Pergamon Press: Oxford, UK (1995);
G. Wilkinson; F. G A. Stone; E. W. Abel, Eds. Comprehensive Organometallic Chemistry; Pergamon Press: Oxford, UK (1982);
B. M. Trost; I. Fleming. Comprehensive Organic Synthesis; Pergamon Press: Oxford, UK (1991);
A. R. Katritzky; C. W. Rees Eds. Comprehensive Heterocylic Chemistry;

Pergamon Press: Oxford, UK (1984);
A. R. Katritzky; C. W. Rees; E. F. V. Scriven, Eds. Comprehensive Heterocylic Chemistry II; Pergamon Press: Oxford, UK (1996); and
C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie (Houben-Weyl); Thieme: Stuttgart, Germany. Furthermore, databases of synthetic transformations include Chemical Abstracts, which may be searched using either CAS OnLine or SciFinder, Handbuch der Organischen Chemie (Beilstein), which may be searched using SpotFire, and REACCS.

The fluoro-substituted diaryl urea Formula (I) has been previously characterized as having various activities, including for inhibiting the VEGFR, PDGFR, raf, p38, and/or flt-3 kinase signaling pathways. These activities and their use in treating various diseases and conditions are disclosed in, e.g., WO/2005/009961, WO/2006/026500, WO/2008/043446, WO/2008/089389, WO/2008/089388, US 2008-0262236, US 2008-02427607, US 2009-0192127, and US 2009-0306020, which are hereby incorporated by reference in their entirety.

Pharmacuetical compositions intended for oral use may be prepared according to any suitable method known to the art for the manufacture of pharmaceutical compositions. The pharmaceutical composition comprises suitable administration forms which deliver the compounds of the drug combinations of this invention in a rapid manner, for example tablets (uncoated or coated tablets), tablets which disintegrate rapidly in the oral cavity or capsules optionally filled with granules (for example hard or soft gelatin capsules), sugar-coated tablets, powders, sachets, granules, pellets, dragées, chewable tablets, dispersible tables, troches and lozenges. Such compositions may contain one or more agents selected from the group consisting of diluents, sweetening agents, flavoring agents, coloring agents and preserving agents in order to provide palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Pharmacuetical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing at least one of the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient.

A pharmaceutically effective amount of compound is that amount which produces a result or exerts an influence on the particular condition being treated. The compounds of the drug combination of present invention can be administered with pharmaceutically-acceptable carriers well known in the art using any effective conventional dosage unit forms, including immediate, slow and timed release preparations.

A pharmaceutically acceptable excipient is any excipient which is relatively non-toxic and innocuous to a patient at concentrations consistent with effective activity of the active ingredient so that any side effects ascribable to the excipient do not vitiate the beneficial effects of the active ingredient.

Pharmaceutically acceptable excipients according to the invention are for example disintegrants, binders, lubricants, fillers, plasticizers, surfactants and wetting agents, film-forming agents and coating materials, and coloring agents for example pigments.

Disintegrants include, but are not limited to croscarmellose sodium, crospovidone, alginic acid, carboxymethylcellulose calcium, carboxymethylcellulose sodium, microcrystalline cellulose, hydroxypropyl cellulose, low substituted hydroxypropyl cellulose, polacrillin potassium, cross-linked polyvinylpyrrolidone, sodium alginate, sodium starch glycollate, partially hydrolysed starch, sodium carboxymethyl starch and starch. Preference is given to croscarmellose sodium and/or cross-linked polyvinylpyrrolidone, more preference is given to croscarmellose sodium.

The amount of the disintegrant contained in the pharmaceutical composition of can be from 0 to 15%, preferably from 5 to 12% by the total weight of the composition.

Binders include, but are not limited to hydroxypropyl cellulose, hypromellose (hydroxypropyl methylcellulose, HPMC), microcrystalline cellulose, acacia, alginic acid, carboxymethylcellulose, ethylcellulose, methylcellulose, hydroxaethylcellulose, ethylhydroxyethylcellulose, polyvinyl alcohol, polyacrylates, carboxymethylcellulose calcium, carboxymethylcellulose sodium, compressible sugar, ethylcellulose, gelatin, liquid glucose, methylcellulose, polyvinyl pyrrolidone and pregelatinized starch. Preference is given to a hydrophilic binder which is soluble in the granulation liquid, more preference is given to hypromellose (hydroxypropyl methylcellulose, HPMC) and/or polyvinylpyrrolidone, most preference is given to hypromellose.

The amount of the binder contained in the pharmaceutical composition of can be from 0 to 15%, preferably from 0.5 to 8% by the total weight of the composition.

Lubricants include, but are not limited to calcium stearate, magnesium stearate, mineral oil, stearic acid, fumaric acid, sodium stearylfumarate, zinc stearate and polyethyleneglycol. Preference is given to magnesium stearate.

The amount of the lubricant contained in the pharmaceutical composition of can be from 0 to 2%, preferably from 0.2 to 0.8% by the total weight of the composition.

Fillers include, but are not limited to dibasic calcium phosphate, kaolin, lactose, mannitol, microcrystalline cellulose, silicated microcrystalline cellulose, dicalcium phosphate, tricalcium phosphate, magnesium trisilicate, mannitol, maltitol, sorbitol, xylitol, lactose for example the anhydrous form or the hydrate form such as the monohydrate form, dextrose, maltose, saccharose, glucose, fructose or maltodextrine, powdered cellulose, precipitated calcium carbonate, sodium carbonate, sodium phosphate and starch. Preference is given to microcrystalline cellulose, mannitol, lactose and/or dicalcium phosphate, more preference is given to microcrystalline cellulose.

The amount of the filler contained in the pharmaceutical composition of can be from 0 to 60%, preferably from 3 to 20% by the total weight of the composition.

Surfactants and Wetting agents include, but are not limited to heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan mono-palmitate, sodium salts of fatty alcohol sulfates such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides. Preference is given to sodium lauryl sulfate.

The amount of the surfactant contained in the pharmaceutical composition of can be from 0 to 5%, preferably from 0.1 to 2% by the total weight of the composition.

Film-forming agents and coating materials include, but are not limited to liquid glucose, hydroxyethyl cellulose, hydroxypropyl cellulose, hydroxypropyl methylcellulose (hypromellose, HPMC), methylcellulose, ethylcellulose, cellulose acetate phthalate, shellac, polyvinylpyrrolidone, copolymers of vinylpyrrolidone and vinylacetate such as Kollidon® VA64 BASF, copolymers of acrylic- and/or methacrylic acid esters with trimethylammoniummethylacrylate, copolymers of dimethylaminomethacrylic acid and neutral methacrylic acid esters, polymers of methacrylic acid or methacrylic acid esters, copolymers of acrylic acid ethylester and methacrylic acid methyl ester, and copolymers of acrylic acid and acrylic acid methylester. Preference is given to hydroxypropyl methylcellulose (hypromellose, HPMC) as film-forming agent.

Plasticizers include, but are not limited to polyethylene glycol, diethyl phthalate and glycerol. Preference is given to polyethylene glycol.

Coloring agents include, but are not limited to pigments, inorganic pigments, FD&C Red No. 3, FD&C Red No. 20, FD&C Yellow No. 6, FD&C Blue No. 2, D&C Green No. 5, D&C Orange No. 5, D&C Red No. 8, caramel, ferric oxide red, ferric oxide yellow and titanium dioxide. Preference is given to ferric oxide red, ferric oxide yellow and titanium dioxide.

Further commonly used pharmaceutical excipients which can be used as appropriate to formulate the composition for its intended route of administration include, but is not limited to: Acidifying agents for example acetic acid, citric acid, fumaric acid, hydrochloric acid and nitric acid; alkalizing agents for example ammonia solution, ammonium carbonate, diethanolamine, monoethanolamine, potassium hydroxide, sodium borate, sodium carbonate, sodium hydroxide, triethanolamine and trolamine; adsorbents for example powdered cellulose and activated charcoal; stabilizers and antioxidants for example ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate and sodium metabisulfite; other binding materials for example block polymers, natural and synthetic rubber, polyacrylates, polyurethanes, silicones, polysiloxanes and styrene-butadiene copolymers; buffering agents for examples potassium metaphosphate, dipotassium phosphate, sodium acetate, sodium citrate anhydrous and sodium citrate hydrates; encapsulating agents for example gelatin, starch and cellulose derivates); flavorants, masking agents and odors for example anise oil, cinnamon oil, cocoa, menthol, orange oil, peppermint oil and vanillin; humectants for example glycerol, propylene glycol and sorbitol; sweeteners for example aspartame, dextrose, glycerol, mannitol, propylene glycol, saccharin sodium, sorbitol and sucrose; anti-adherents for example magnesium stearate and talc; direct compression excipients for example dibasic calcium phosphate, lactose and microcrystalline cellulose; tablet polishing agents for example carnauba wax and white wax.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnauba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

The component of the drug combination comprising the compound of Formula I can be in the form of solid dispersion with a pharmaceutically acceptable matrix. The solid dispersion can be one of the different types as defined in WO 2006/26500 such as: solid solutions, glass solutions, glass suspensions, amorphous precipitations in a crystalline carrier, eutectics or monotecics, compound or complex formation and combinations thereof.

The pharmaceutically acceptable matrix preferably comprises a pharmaceutically acceptable polymer, such as, for example, polyvinylpyrrolidone, vinylpyrrolidone/vinylacetate copolymer, crospovidone, polyalkylene glycol (e.g. polyethylene glycol), polyethylenoxide, poloxamer, hydroxyalkyl cellulose (e.g. hydroxypropyl cellulose), hydroxyalkyl methyl cellulose (e.g. hydroxypropyl methyl cellulose), carboxymethyl cellulose, sodium carboxymethyl cellulose, ethyl cellulose, cellulose succinates (e.g. cellulose acetate succinate and hydroxypropyl methyl cellulose acetate succinate), cellulose phthalates (e.g. cellulose acetate phthalate and hydroxypropyl methyl cellulose phthalate), polymethacrylates (e.g. Eudragit® types), polyhydroxyalkylacrylates, polyhydroxyalkylmethacrylates, polyacrylates, polyvinyl alcohol, polyvinyl acetate, vinyl alcohol/vinyl acetate copolymer, xanthan gum, galactomannanes, carrageenan, chitosan, chitin, alginic acid and its salts, polylactides, dextrins, starch and starch derivatives, proteins and combinations thereof.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

Micronization of the powders and granules can be achieved by standard milling methods, preferably by air chat milling, known to a skilled person. The micronized form can have a mean particle size of from 0.5 to 10 μm, preferably from 1 to 6 μm, more preferably from 1 to 3 μm. The indicated particle size is the mean of the particle size distribution measured by laser diffraction known to a skilled person (measuring device: HELOS, Sympatec).

The components of the drug combination may also be in the form of non-aqueous liquid formulations, e.g., oily suspensions which may be formulated by suspending the active ingredients in polyethyleneglycol, a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

The components of the drug combination of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The active ingredients of the drug combination of the invention may also be administered transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of a fluoro-substituted diaryl urea of Formula (I) in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of an aryl urea compound may be formulated into a lotion or salve.

Suitable solvents for processing transdermal delivery systems are known to those skilled in the art, and include dimethylsulfoxide, lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery systems are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

Specific preparations of compounds within the drug combination of this invention can be adapted from others known in the art. For example, Riedl, B., et al., “O-Carboxy Aryl Substituted Diphenyl Ureas as raf Kinase Inhibitors” PCT Int. Appl., WO 00 42012, Riedl, B., et al., “O-Carboxy Aryl Substituted Diphenyl Ureas as p38 Kinase Inhibitors” PCT Int. Appl., WO 00 41698, incorporated herein by reference.

Pharmaceutical compositions according to the present invention can be illustrated as follows:

Sterile IV Solution: A 5 mg/ml solution of a desired compound of the drug combination of this invention is made using sterile, injectable water, and the pH is adjusted if necessary. The solution is diluted for administration to 1-2 mg/ml with sterile 5% dextrose and is administered as an IV infusion over 60 minutes.

Lyophilized powder for IV administration: A sterile preparation can be prepared with (i) 100-1000 mg of a desired compound of the drug combination of this invention as a lyophilized powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.

Intramuscular suspension: The following solution or suspension can be prepared, for intramuscular injection:
50 mg/ml of the desired, water-insoluble compound of the drug combination of this invention
5 mg/ml sodium carboxymethylcellulose
4 mg/ml TWEEN 80
9 mg/ml sodium chloride
9 mg/ml benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid-state extraction techniques. The drug compounds may be compressed with viscoelastic and thermoelastic sugars and polymers or effervescent components to produce porous matrices intended for immediate release, without the need of water.

The invention also encompasses kits for treating mammalian cancers. Such kits can be used to treat a patient with a raf kinase stimulated cancer as well as cancers not stimulated through raf kinase. The kit can comprise a single pharmaceutical formulation containing a fluoro-substituted diaryl urea compound of Formula I (e.g., Regorafenib) and antifolate, (e.g., Pemetrexed) and optionally a platinum complex (e.g., cisplatin). Alternatively, the kit can comprise a fluoro-substituted diaryl urea compound of Formula I an antifolate and platinum complex in separate formulations. The kit can also include instructions for how to administer the compounds to a patient with cancer in need of treatment. The kit can be used to treat different cancer types which include but are not limited to NSCLC, colon, prostate, leukemia, melanoma, hepatocellular, renal, head and neck, glioma, lung, pancreatic, ovarian, and mammary.

It will be appreciated by those skilled in the art that the particular method of administration will depend on a variety of factors, all of which are routinely considered when administering therapeutics. It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of one or more of the drugs within the combination (or a pharmaceutically acceptable salt thereof) given for a defined number of days, can be ascertained by those skilled in the art using conventional treatment tests.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and gender of the patient treated, and the nature and extent of the condition treated.

Generally, the use of the drug combination of this invention will serve to (1) yield better efficacy in reducing the growth of a tumor or even eliminate the tumor as compared to administration of a single chemotherapeutic agent, (2) provide for the administration of lesser amounts of the administered chemotherapeutic agents, (3) provide for a chemotherapeutic treatment that is well tolerated in the patient with less deleterious pharmacological complications resulting from larger doses of single chemotherapies and certain other combined therapies, (4) provide for treating a broader spectrum of different cancer types in mammals, especially humans, (5) provide for a higher response rate among treated patients, (6) provide for a longer survival time among treated patients compared to standard chemotherapy treatments, (7) provide a longer time for tumor progression, and/or (8) yield efficacy and tolerability results at least as good as those of the agents used alone, compared to known instances where other cancer agent combinations produce antagonist effects.

The fluoro-substituted diaryl urea compound of Formula (I), or polymorphs, solvates, hydrates, metabolites, prodrugs, pharmaceutically acceptable salts or diastereoisomers thereof, can be administered to a patient at a dosage which can range from about 0.001 to about 300 mg/Kg of total body weight, typically about 160 mg/Kg of total body weight. A unit dosage may contain from about 0.5 mg to about 2000 mg of active ingredient, and can be administered one or more times per day. Preference is given to an amount of the fluoro-substituted diaryl urea compound of Formula (I) (or polymorphs, solvates, hydrates, metabolites, prodrugs, pharmaceutically acceptable salts or diastereoisomers thereof) in the pharmaceutical composition from 20 to 2000 mg, preferably from 40 to 800 mg, more preferably from 50 to 600 mg. Particular preference is given to an amount in the pharmaceutical composition from 27 to 2740 mg, preferably from 54 to 1096, more preferably from 68 to 822 mg.

The daily dose for oral administration will preferably be from 0.1 to 300 mg/kg of total body weight, typically from about 0.1 mg/kg to about 50 mg/kg body weight per day. The daily dosage for administration by injection which includes intravenous, intramuscular, subcutaneous and parenteral injection as well as infusion techniques will preferably be from 0.1 to 300 mg/kg of total body weight. The daily vaginal dosage regime will preferably be from 0.1 to 300 mg/kg of total body weight. The daily topical dosage regimen will preferably be from 0.1 to 300 mg administered between one to four times daily. The transdermal concentration will preferably be that required to maintain a daily dose of from 1 to 300 mg/kg. For all the above mentioned routes of administration, the preferred dosage is 0.1 to 300 mg/kg. The daily inhalation dosage regimen will preferably be from 0.1 to 300 mg/kg of total body weight.

The antifolates such as Pemetrexed and platinum complexes such as cisplatin are each preferably administered non-orally, more preferably by intravenous infusion, in conventional amounts routinely used in cancer monotherapy or reduced amounts, based on the combination of active agents.

Based on body surface area and the frequency of dosage, the infusion dosage of the antifolates such as Pemetrexed may range from about 10 to above 500 mg/m², preferably about 500 mg/m² for a single dose. Antifolate infusions should be preceded with appropriate premedications known to those skilled in the art.

The platinum complex dosage is preferably administered intravenously by infusion over a period of at least about 3 hours, preferably over a period of about 3 or 24 hours and may be administered at separate intervals over a course of 5 days. A single dose intended for a 3-4 week period can range from 50 to 100 mg/m² (patient surface area). A daily dose of 15 to 20 mg/m² for 5 days every 3 to 4 weeks is an alternative to a single dose.

For each of the fluoro-substituted diaryl urea compound of Formula (I), antifolate and platinum complex, the administered dosage of the compound may be modified depending on any superior or unexpected results which may be obtained as routinely determined with this invention. Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the pharmaceutical compositions of this invention can readily be determined by those skilled in the art. The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

The fluoro-substituted diaryl urea compound of Formula (I), polymorphs, solvates, hydrates, metabolites, prodrugs, pharmaceutically acceptable salts or diastereoisomers thereof can be administered orally, topically, parenterally, rectally, by inhalation, and by injection. Administration by injection includes intravenous, intramuscular, subcutaneous, and parenterally as well as by infusion techniques. The fluoro-substituted aryl urea compound of Formula (I), polymorphs, solvates, hydrates, metabolites, prodrugs, pharmaceutically acceptable salts or diastereoisomers thereof can be present in association with one or more non-toxic pharmaceutically acceptable carriers and if desired other active ingredients. A preferred route of administration for the aryl urea compound is oral administration.

The antifolates and platinum complexes can be administered to a patient by any of the conventional routes of administration for these compounds. This can include oral, topical, parenteral, rectal and inhalation administration as well as injection. Administration by injection includes intravenous, intramuscular, subcutaneous, and parenterally as well as by infusion techniques. The preferred route of administration for the antifolates and platinum complexes used in this invention is typically by injection which is the same route of administration used for the agent alone. Any of the antifolates and platinum complexes can be administered in combination with a fluoro-substituted diaryl urea compound of Formula (I) (or polymorphs, solvates, hydrates, metabolites, prodrugs, pharmaceutically acceptable salts or diastereoisomers thereof) by any of the mentioned routes of administration.

For administering the fluoro-substituted diaryl urea compound of Formula (I) and both the antifolates and platinum complexes, by any of the routes of administration herein discussed, the fluoro-substituted diaryl urea compound of Formula (I) (or polymorphs, solvates, hydrates, metabolites, prodrugs, pharmaceutically acceptable salts or diastereoisomers thereof) can be administered simultaneously with the antifolates and platinum complexes. This can be performed by administering a single formulation which contains both the fluoro-substituted diaryl urea compound of Formula (I) (polymorphs, solvates, hydrates, metabolites, prodrugs, pharmaceutically acceptable salts or diastereoisomers thereof) and the antifolates and platinum complex or administering the fluoro-substituted diaryl urea compound of Formula (I) and the antifolates and platinum complex in independent formulations at the same time to a patient.

Alternatively, the fluoro-substituted diaryl urea compound of Formula (I) (or polymorphs, solvates, hydrates, metabolites, prodrugs, pharmaceutically acceptable salts or diastereoisomers thereof) can be administered in tandem with the antifolates and optionally the platinum complex. The fluoro-substituted diaryl urea compound of Formula (I) (or polymorphs, solvates, hydrates, metabolites, prodrugs, pharmaceutically acceptable salts or diastereoisomers thereof) can be administered prior to either the antifolates and (optionally) the platinum complex or both. For example, the fluoro-substituted aryl urea compound of Formula (I) (or polymorphs, solvates, hydrates, metabolites, prodrugs, pharmaceutically acceptable salts or diastereoisomers thereof) can be administered one or more times per day up to 28 consecutive days followed by administration of the antifolates and optional platinum complex. Also, either the antifolates and optional the platinum complex or both can be administered first followed by administration of the fluoro-substituted diaryl urea compound of Formula (I). The choice of sequence administration of the fluoro-substituted diaryl urea compound of Formula (I) (or polymorphs, solvates, hydrates, metabolites, prodrugs, pharmaceutically acceptable salts or diastereoisomers thereof) relative to the antifolates and optional platinum complex may vary for different agents. Also, the antifolates and optional platinum complex can be administered using any regimen which is conventionally used for these agents.

In another regimen of administration, the fluoro-substituted diaryl urea compound of Formula (I) (or polymorphs, solvates, hydrates, metabolites, prodrugs, pharmaceutically acceptable salts or diastereoisomers thereof) and the antifolates and optional platinum complex can be administered one or more times per day on the day of administration.

Methods of Use

The present invention provides drug combinations which are capable of modulating one or more signal transduction pathways involving raf, VEGFR, PDGFR, p38, and/or flt-3 kinases. Raf is an important signaling molecule involved in the regulation of a number of key cellular processes, including cell growth, cell survival and invasion. It is a member of the Ras/raf/MEK/ERK pathway. This pathway is present in most tumor cells. VEGFR, PDGFR, and flt-3 are transmembrane receptor molecules which, when stimulated by an appropriate ligand, trigger the Ras/raf/MEK/ERK cell signaling pathway, leading to a cascade of cellular events. Each of these receptor molecules have tyrosine kinase activity.

The VEGFR receptors are stimulated by vascular endothelial growth factors (VEGF), and are important control points in the regulation of endothelial cell development and function. The PDGF-beta receptor regulates cell proliferation and survival in a number of cell types, including mesenchymal cells. Flt-3 is a receptor for the FL ligand. It is structurally similar to c-kit, and modulates the growth of pluripotent haemopoietic cells, influencing the development of T-cells, B-cells, and dendritic cells.

Any gene or isoform of raf, VEGFR, PDGFR, p38, and/or flt-3 can be modulated in accordance with present invention, including both wild-type and mutant forms. Raf or raf-1 kinase is a family of serine/threonine kinases which comprise at least three family members, a-raf, b-raf, and c-raf or raf-1. See, e.g., Dillon and Kolch, Arch. Biochem. Biophys. 2002, 404, 3-9. C-raf and b-raf are preferred targets for compounds of the present invention. Activating b-raf mutations (e.g., V599E mutant) have been identified in various cancers, including melanoma, and the compounds described herein can be utilized to inhibit their activity.

By the term “modulate”, it is meant that the functional activity of the pathway (or a component of it) is changed in comparison to its normal activity in the absence of the compound. This effect includes any quality or degree of modulation, including, increasing, agonizing, augmenting, enhancing, facilitating, stimulating, decreasing, blocking, inhibiting, reducing, diminishing, antagonizing, etc.

The drug combinations of the present invention can also modulate one or more of the following processes, including, but not limited to, e.g., cell growth (including, e.g., differentiation, cell survival, and/or proliferation), tumor cell growth (including, e.g., differentiation, cell survival, and/or proliferation), tumor regression, endothelial cell growth (including, e.g., differentiation, cell survival, and/or proliferation), angiogenesis (blood vessel growth), lymphangiogenesis (lymphatic vessel growth), and/or hematopoiesis (e.g., T- and B-cell development, dendritic cell development, etc.).

While not wishing to be bound by any theory or mechanism of action, it has been found that compounds of the drug combination of present invention possess the ability to modulate kinase activity. The methods of the present invention, however, are not limited to any particular mechanism or how the drug combinations achieve their therapeutic effect. By the term “kinase activity”, it is meant a catalytic activity in which a gamma-phosphate from adenosine triphosphate (ATP) is transferred to an amino acid residue (e.g., serine, threonine, or tyrosine) in a protein substrate. A compound of the drug combination can modulate kinase activity, e.g., inhibiting it by directly competing with ATP for the ATP-binding pocket of the kinase, by producing a conformational change in the enzyme's structure that affects its activity (e.g., by disrupting the biologically-active three-dimensional structure), etc.

Kinase activity can be determined routinely using conventional assay methods. Kinase assays typically comprise the kinase enzyme, substrates, buffers, and components of a detection system. A typical kinase assay involves the reaction of a protein kinase with a peptide substrate and an ATP, such as ³²P-ATP, to produce a phosphorylated end-product (for instance, a phosphoprotein when a peptide substrate is used). The resulting end-product can be detected using any suitable method. When radioactive ATP is utilized, a radioactively labeled phosphoprotein can be separated from the unreacted gamma-³²P-ATP using an affinity membrane or gel electrophoresis, and then visualized on the gel using autoradiography or detected with a scintillation counter. Non-radioactive methods can also be used. Methods can utilize an antibody which recognizes the phosphorylated substrate, e.g., an anti-phosphotyrosine antibody. For instance, kinase enzyme can incubated with a substrate in the presence of ATP and kinase buffer under conditions which are effective for the enzyme to phosphorylate the substrate. The reaction mixture can be separated, e.g., electrophoretically, and then phosphorylation of the substrate can be measured, e.g., by Western blotting using an anti-phosphotyrosine antibody. The antibody can be labeled with a detectable label, e.g., an enzyme, such as HRP, avidin or biotin, chemiluminescent reagents, etc. Other methods can utilize ELISA formats, affinity membrane separation, fluorescence polarization assays, luminescent assays, etc.

An alternative to a radioactive format is time-resolved fluorescence resonance energy transfer (TR-FRET). This method follows the standard kinase reaction, where a substrate, e.g., biotinylated poly(GluTyr), is phosphorylated by a protein kinase in the presence of ATP. The end-product can then detected with a europium chelate phosphospecific antibody (anti-phosphotyrosine or phosphoserine/threonine), and streptavidin-APC, which binds the biotinylated substrate. These two components are brought together spatially upon binding, and energy transfer from the phosphospecific antibody to the acceptor (SA-APC) produces fluorescent readout in the homogeneous format.

The drug combinations of the present invention can be used to treat and/or prevent any disease or condition mediated by one or more cellular signal transduction pathways involving raf, VEGFR, PDGFR, p38, and/or flt-3 kinases. The term “treating” is used conventionally, e.g., the management or care of a subject for the purpose of combating, alleviating, reducing, relieving, improving the condition of, etc., of a disease or disorder. The drug combinations can also be described as being used to prevent and/or treat diseases and/or condition mediated by the signaling molecules. The term “mediated” indicates, e.g., that the signaling molecule is part of the pathway which is aberrant or disturbed in the disease and/or condition.

Diseases and conditions that can be treated include any of those mentioned above and below, as well as:

Raf associated diseases include, e.g., cell-proliferation disorders, cancer, tumors, etc.;

VEGFR-2 associated diseases include, e.g., cancer, tumor growth, inflammatory disease, rheumatoid arthritis, retinopathy, psoriasis, glomerulonephritis, asthma, chronic bronchitis, atherosclerosis, transplant rejection, conditions involving angiogenesis, etc.;

VEGFR-3 associated diseases include, e.g., cancer, corneal disease, inflamed cornea (e.g., Hamrah, Am. J. Path. 2003, 163, 57-68), corneal transplantation (Cursiefen et al., Cornea 2003, 22, 273-81), lymphatic hyperplasia, conditions involving lymphangiogenesis, etc.;

PDGFR-beta associated diseases include, e.g., diseases or conditions characterized by cell proliferation, cell matrix production, cell movement, and/or extra cellular matrix production. Specific examples, include, e.g., tumors, malignancies, cancer, metastasis, chronic myeloid leukemia, inflammation, renal disease, diabetic nephropathy, mesangial proliferative glomerulonephritis, fibrotic conditions, atherosclerosis, restenosis, hypertension-related arteriosclerosis, venous bypass graft arteriosclerosis, scleroderma, interstitial pulmonary diseases, synovial disorders, arthritis, leukemias, lymphomas, etc;

Flt-3 associated diseases include, e.g., immune-related disorders, blood cell disorders, conditions involving hematopoietic cell development (e.g., T-cells, B-cells, dendritic cells, cancer, anemia, HIV, acquired immune deficiency syndrome, etc.

p38 associated diseases include inflammatory disorders, immunomodulatory disorders, and other disorders that have been linked to abnormal cytokine production, especially TNF-alpha, or abnormal MMP activity. These disorders include, but are not limited to, rheumatoid arthritis, COPD, osteoporosis, Crohn's disease and psoriasis.

In addition, drug combinations of the present invention can be used to treat conditions and disorders disclosed in U.S. Pat. No. 6,316,479, e.g., glomerular sclerosis, interstitial nephritis, interstitial pulmonary fibrosis, atherosclerosis, wound scarring and scleroderma.

The following publications relate to VEGFR-3 modulation and are incorporated herein for their description of disease states mediated by VEGFR-3 and assays to determine such activity.

| | |
|---|---|
| WO95/33772 | Alitalo, et. al. |
| WO95/33050 | Charnock-Jones, et. al.. |
| WO96/39421 | Hu, et. al. |
| WO98/33917 | Alitalo, et. al. |
| WO02/057299 | Alitalo, et. al. |
| WO02/060950 | Alitalo, et. al. |
| WO02/081520 | Boesen, et. al. |

The following publications relate to VEGFR-2 modulation and are incorporated herein for their description of disease states mediated by VEGFR-2 and assays to determine such activity.

| | |
|---|---|
| EP0882799 | Hanai, et. al. |
| EP1167384 | Ferraram, et, al. |
| EP1086705 | Sato, et. al. |
| EP11300032 | Tesar, et. al. |
| EP1166798 | Haberey, et. al. |
| EP1166799 | Haberey, et. al. |
| EP1170017 | Maini, et. al. |
| EP1203827 | Smith |
| WO02/083850 | Rosen, et. al. |

The following publications relate to flt-3 modulation and are incorporated herein for their description of disease states mediated by flt-3 and assays to determine such activity.

| | |
|---|---|
| 2002/0034517 | Brasel, et. al. |
| 2002/0107365 | Lyman, et. al. |
| 2002/0111475 | Graddis, et. al. |
| EP0627487 | Beckermann, et. al. |
| WO9846750 | Bauer, et. al. |
| WO9818923 | McWherter, et. al. |
| WO9428391 | Beckermann, et al. |
| WO9426891 | Birnbaum, et. al. |

The following patents and publication relate to PDGF/PDGFR modulation and are incorporated herein for their description of the disease states mediated by PDGFR-beta and assays to determine such activity.

| | |
|---|---|
| 5,094,941 | Hart, et. al. |
| 5,371,205 | Kelly, et. al. |
| 5,418,135 | Pang |
| 5,444,151 | Vassbotn, et. al. |
| 5,468,468 | LaRochelle, et. al. |
| 5,567,584 | Sledziewski, et. al. |
| 5,618,678 | Kelly, et. al. |
| 5,620,687 | Hart, et. al. |
| 5,648,076 | Ross, et. al. |
| 5,668,264 | Janjic, et. al. |
| 5,686,572 | Wolf, et. al. |
| 5,817,310 | Ramakrishnan, et. al. |
| 5,833,986 | LaRochelle, et. al. |
| 5,863,739 | LaRochelle, et. al. |
| 5,872,218 | Wolf, et. al. |
| 5,882,644 | Chang, et. al. |
| 5,891,652 | Wolf, et. al. |
| 5,976,534 | Hart, et. al. |
| 5,990,141 | Hirth, et. al. |
| 6,022,854 | Shuman |
| 6,043,211 | Williams, et. al. |
| 6,110,737 | Escobedo, et. al. |
| 6,207,816B1 | Gold, et. al. |
| 6,228,600B1 | Matsui, et. al. |
| 6,229,002B1 | Janjic, et. al. |
| 6,316,603B1 | McTigue, et. al. |
| 6,372,438B1 | Williams, et. al. |
| 6,403,769B1 | La Rochelle, et. al. |
| 6,440,445B1 | Nowak, et. al. |
| 6,475,782B1 | Escobedo, et. al. |
| WO02/083849 | Rosen, et. al. |
| WO02/083704 | Rosen, et. al. |
| WO02/081520 | Boesen, et. al. |
| WO02/079498 | Thomas, et. al. |
| WO02/070008 | Rockwell, et. al. |
| WO09959636 | Sato, et. al. |
| WO09946364 | Cao, et. al. |
| WO09940118 | Hanai, et. al. |
| WO9931238 | Yabana, et. al. |
| WO9929861 | Klagsbrun, et. al. |
| WO9858053 | Kendall, et. al. |
| WO9851344 | Maini, et. al. |
| WO9833917 | Alitalo, et. al. |
| WO9831794 | Matsumoto, et. al. |
| WO9816551 | Ferrara, et. al. |
| WO9813071 | Kendall, et al. |
| WO9811223 | Martiny-Baron, et. al. |
| WO9744453 | Chen, et. al. |
| WO9723510 | Plouet, et. al. |
| WO9715662 | Stinchcomb, et. al. |
| WO9708313 | Ferrara, et. al. |
| WO9639515 | Cao, et. al. |
| WO9623065 | Smith, et. al. |
| WO9606641 | Fleurbaaij, et. al. |
| WO9524473 | Cao, et. al. |
| WO9822316 | Kyowa |
| WO9521868 | Rockwell, et. al. |
| WO02/060489 | Xia, et. al. |

PDGFR-Beta

| | |
|---|---|
| EP0869177 | Matsui, et. al. |
| WO09010013 | Matsui, et. al. |
| WO9737029 | Matsui, et. al. |

PDGFR-Alpha

| | |
|---|---|
| EP1000617 | Lammers, et. al. |
| EP0869177 | Matsui, et. al. |
| EP0811685 | Escobedo, et. al. |

The drug combinations of this invention also have a broad therapeutic activity to treat or prevent the progression of a broad array of diseases, such as inflammatory conditions, coronary restenosis, tumor-associated angiogenesis, atherosclerosis, autoimmune diseases, inflammation, certain kidney diseases associated with proliferation of glomerular or mesangial cells, and ocular diseases associated with retinal vessel proliferation, psoriasis, hepatic cirrhosis, diabetes, atherosclerosis, restenosis, vascular graft restenosis, in-stent stenosis, angiogenesis, ocular diseases, pulmonary fibrosis, obliterative bronchiolitis, glomerular nephritis, rheumatoid arthritis.

The present invention also provides for treating, preventing, modulating, etc., one or more of the following conditions in humans and/or other mammals: retinopathy, including diabetic retinopathy, ischemic retinal-vein occlusion, retinopathy of prematurity and age related macular degeneration; rheumatoid arthritis, psoriasis, or bullous disorder associated with subepidermal blister formation, including bullous pemphigoid, erythema multiforme, or dermatitis herpetiformis, rheumatic fever, bone resorption, postmenopausal osteoporosis, sepsis, gram negative sepsis, septic shock, endotoxic shock, toxic shock syndrome, systemic inflammatory response syndrome, inflammatory bowel disease (Crohn's disease and ulcerative colitis), Jarisch-Herxheimer reaction, asthma, adult respiratory distress syndrome, acute pulmonary fibrotic disease, pulmonary sarcoidosis, allergic respiratory disease, silicosis, coal worker's pneumoconiosis, alveolar injury, hepatic failure, liver disease during acute inflammation, severe alcoholic hepatitis, malaria (Plasmodium falciparum malaria and cerebral malaria), non-insulin-dependent diabetes mellitus (NIDDM), congestive heart failure, damage following heart disease, atherosclerosis, Alzheimer's disease, acute encephalitis, brain injury, multiple sclerosis (demyelation and oligiodendrocyte loss in multiple sclerosis), advanced cancer, lymphoid malignancy, pancreatitis, impaired wound healing in infection, inflammation and cancer, myelodysplastic syndromes, systemic lupus erythematosus, biliary cirrhosis, bowel necrosis, radiation injury/toxicity following administration of monoclonal antibodies, host-versus-graft reaction (ischemia reperfusion injury and allograft rejections of kidney, liver, heart, and skin), lung allograft rejection (obliterative bronchitis), or complications due to total hip replacement, ad an infectious disease selected from tuberculosis, Helicobacter pylori infection during peptic ulcer disease, Chaga's disease resulting from Trypanosoma cruzi infection, effects of Shiga-like toxin resulting from E. coli infection, effects of enterotoxin A resulting from Staphylococcus infection, meningococcal infection, and infections from Borrelia burgdorferi, Treponema pallidum, cytomegalovirus, influenza virus, Theiler's encephalomyelitis virus, and the human immunodeficiency virus (HIV), papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, colorectal cancer, thyroid cancer, pancreatic cancer, gastric cancer, hepatocellular carcinoma, leukemia, lymphoma, Hodgkin's disease, Burkitt's disease, arthritis, rheumatoid arthritis, diabetic retinopathy, angiogenesis, restenosis, in-stent restenosis, vascular graft restenosis, pulmonary fibrosis, hepatic cirrhosis, atherosclerosis, glomerulonophritis, diabetic nephropathy, thrombic micoangiopathy syndromes, transplant rejection, psoriasis, diabetes, wound healing, inflammation, and neurodegenerative diseases, hyperimmune disorders, hemangioma, myocardial angiogenesis, coronary and cerebral collateral vascularization, ischemia, corneal disease, rubeosis, neovascular glaucoma, macular degeneration retinopathy of prematurity, wound healing, ulcer Helicobacter related diseases, fractures, endometriosis, a diabetic condition, cat scratch fever, thyroid hyperplasia, asthma or edema following burns, trauma, chronic lung disease, stroke, polyps, cysts, synovitis, chronic and allergic inflammation, ovarian hyperstimulation syndrome, pulmonary and cerebral edema, keloid, fibrosis, cirrhosis, carpal tunnel syndrome, adult respiratory distress syndrome, ascites, an ocular condition, a cardiovascular condition, Crow-Fukase (POEMS) disease, Crohn's disease, glomerulonophritis, osteoarthritis, multiple sclerosis, graft rejection, Lyme disease, sepsis, von Hippel Lindau disease, pemphigoid, Paget's disease, polycystic kidney disease, sarcoidosis, throiditis, hyperviscosity syndrome, Osler-Weber-Rendu disease, chronic occlusive pulmonary disease, radiation, hypoxia, preeclampsia, menometrorrhagia, endometriosis, infection by Herpes simplex, ischemic retinopathy, corneal angiogenesis, Herpes Zoster, human immunodeficiency virus, parapoxvirus, protozoa, toxoplasmosis, and tumor-associated effusions and edema.

The drug combinations of this invention can possess more than one of the mentioned activities, and therefore can target a plurality of signal transduction pathways. Thus, these compounds can achieve therapeutic and prophylactic effects which normally are only obtained when using a combination of different compounds. For instance, the ability to inhibit both new vessel formation (e.g., associated with VEGFR-2 and VEGFR-3 function) (e.g., blood and/or lymph) and cell-proliferation (e.g., associated with raf and PDGFR-beta function) is especially beneficial in the treatment of cancer, and other cell-proliferation disorders that are facilitated by neo-vascularization. Any disorder or condition that would benefit from inhibiting vessel growth and cell proliferation can be treated in accordance with the present invention.

As indicated above, the present invention relates to methods of treating and/or preventing diseases and conditions; and/or modulating one or more of the pathways, polypeptides, genes, diseases, conditions, etc., associated with raf, VEGFR, PDGFR, p38, and/or flt-3. These methods generally involve administering effective amounts of compounds of the drug combination of the present invention, where an effective amount is the quantity of the compounds which is useful to achieve the desired result. The compounds of the drug combination can be administered in any effective form by any effective route, as discussed in more detail below.

Methods include modulating tumor cell proliferation, including inhibiting cell proliferation. The latter indicates that the growth and/or differentiation of tumor cells is reduced, decreased, diminished, slowed, etc. The term “proliferation” includes any process which relates to cell growth and division, and includes differentiation and apoptosis. As discussed above, raf kinases play a key role in the activation of the cytoplasmic signaling cascade involved in cell proliferation, differentiation, and apoptosis. For example, studies have found that inhibiting c-raf by anti-sense oligonucleotides can block cell proliferation (see above). Any amount of inhibition is considered therapeutic.

Included in the methods of the present invention is a method for using the drug combinations described above to treat mammalian hyper-proliferative disorders comprising administering to a mammal, including a human in need thereof, a drug combination of this invention, which is effective to treat the disorder.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Any tumor or cancer can be treated, including, but not limited to, cancers having one or more mutations in raf, ras, and/or flt-3, as well as any upstream or downstream member of the signaling pathways of which they are a part. As discussed earlier, a cancer can be treated with a drug combination of the present invention irrespective of the mechanism which is responsible for it. Cancers of any organ can be treated, including cancers of, but are not limited to, e.g., colon, pancreas, breast, prostate, bone, liver, kidney, lung, testes, skin, pancreas, stomach, colorectal cancer, renal cell carcinoma, hepatocellular carcinoma, melanoma, etc.

### Method of Treating Hyper-Proliferative Disorders Such as Cancer

While the drug combinations of the present invention can be utilized to treat any diseases or conditions that are associated with, or mediated by, the cellular pathways modulated by the compounds therein, of particular interest are methods for using the drug combination according to the invention to treat mammalian hyper-proliferative disorders, including cancer. This method comprises administering a pharmaceutical composition comprising the drug combination to a mammal in need thereof, including a human, an amount which is effective to treat the disorder. The present invention includes any ameliorative or therapeutic effect, regardless of the mechanism of action or how it is achieved.

In treating hyper-proliferative disorders, the drug combination can have one or more of the following activities, including, anti-proliferative; anti-tumor; anti-angiogenic; inhibiting the proliferation of endothelial or tumor cells; anti-neoplastic; immunosuppressive; immunomodulatory; apoptosis-promoting, etc.

Cancers that can be treated in accordance with the present invention include, especially, but not limited to, brain tumors, breast cancer, bone sarcoma (e.g., osteosarcoma and Ewings sarcoma), bronchial premalignancy, endometrial cancer, glioblastoma, hematologic malignancies, hepatocellular carcinoma, Hodgkin's disease, gastrointestinal stromal tumors (G.I.S.T.), kidney neoplasms, leukemia, leimyosarcoma, liposarcoma, lymphoma, Lhermitte-Duclose disease, malignant glioma, melanoma, malignant melanoma, metastases, multiple myeloma, myeloid metaplasia, myeloplastic syndromes, non-small cell lung cancer, pancreatic cancer, prostate cancer, renal cell carcinoma (e.g., advanced, advanced refractory), rhabdomyosarcoma, soft tissue sarcoma, squamous epithelial carcinoma of the skin, thyroid cancer, cancers associated with loss of function of PTEN; activated Akt (e.g. PTEN null tumors and tumors with ras mutations).

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell, non-small-cell lung carcinoma, bronchial adenoma, and pleuropulmonary blastoma.

Examples of brain cancers include, but are not limited to, brain stem and hypophtalmic glioma, cerebellar and cerebral astrocytoma, medulloblastoma, ependymoma, and neuroectodermal and pineal tumor.

Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus.

Tumors of the digestive tract include, but are not limited to, anal, colon, colorectal, esophageal, gallbladder, gastric, pancreatic, rectal, small intestine, salivary gland cancers and gastrointestinal stromal tumors (G.I.S.T.).

Tumors of the urinary tract include, but are not limited to, bladder, penile, kidney, renal pelvis, ureter, and urethral cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, and/or oropharyngeal cancers, and lip and oral cavity cancer.

Thyroid cancers include, but are not limited to, papillary and/or mixed papillary/follicular, follicular and/or Hurthle cell, medullary and anaplastic.

Lymphomas include, but are not limited to, AIDS-related lymphoma, non-Hodgkin's lymphoma, cutaneous T-cell lymphoma, Hodgkin's disease, and lymphoma of the central nervous system.

Sarcomas include, but are not limited to, sarcoma of the soft tissue, osteosarcoma, malignant fibrous histiocytoma, lymphosarcoma and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

In addition to inhibiting the proliferation of tumor cells, the pharmaceutical compositions (drug combinations) of the present invention can also cause tumor regression, e.g., a decrease in the size of a tumor, or in the extent of cancer in the body.

The components of the drug combination can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy, e.g., in having more potent antiproliferative and pro-apoptotic action.

The drug combination of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, pharmaceutical compositions (drug combinations) of the present invention can be administered with other active agents or therapies (e.g., radiation) that are utilized to treat any of the above-mentioned diseases and/or conditions.

The present invention also relates to methods of modulating angiogenesis and/or lymphangiogenesis in a system comprising cells, comprising administering to the system an effective amount of a drug combination described herein. A system comprising cells can be an in vivo system, such as a tumor in a patient, isolated organs, tissues, or cells, in vitro assays systems (CAM, BCE, etc), animal models (e.g., in vivo, subcutaneous, cancer models), hosts in need of treatment (e.g., hosts suffering from diseases having angiogenic and/or lymphangiogenic component, such as cancer), etc.

Inappropriate and ectopic expression of angiogenesis can be deleterious to an organism. A number of pathological conditions are associated with the growth of extraneous blood vessels. These include, e.g., diabetic retinopathy, neovascular glaucoma, psoriasis, retrolental fibroplasias, angiofibroma, inflammation, etc. In addition, the increased blood supply associated with cancerous and neoplastic tissue, encourages growth, leading to rapid tumor enlargement and metastasis. Moreover, the growth of new blood and lymph vessels in a tumor provides an escape route for renegade cells, encouraging metastasis and the consequence spread of the cancer.

Useful systems for measuring angiogenesis and/or lymphangiogenesis, and inhibition thereof, include, e.g., neovascularization of tumor explants (e.g., U.S. Pat. Nos. 5,192,744; 6,024,688), chicken chorioallantoic membrane (CAM) assay (e.g., Taylor and Folkman, Nature 1982, 297, 307-312; Eliceiri et al., J. Cell Biol. 1998, 140, 1255-1263), bovine capillary endothelial (BCE) cell assay (e.g., U.S. Pat. No. 6,024,688; Polverini, P. J. et al., Methods Enzymol. 1991, 198, 440-450), migration assays, and HUVEC (human umbilical cord vascular endothelial cell) growth inhibition assay (e.g., U.S. Pat. No. 6,060,449), and use of the rabbit ear model (e.g., Szuba et al., FASEB J. 2002, 16(14), 1985-7).

Modulation of angiogenesis can be determined by any other method. For example, the degree of tissue vascularity is typically determined by assessing the number and density of vessels present in a given sample. For example, microvessel density (MVD) can be estimated by counting the number of endothelial clusters in a high-power microscopic field, or detecting a marker specific for microvascular endothelium or other markers of growing or established blood vessels, such as CD31 (also known as platelet-endothelial cell adhesion molecule or PECAM). A CD31 antibody can be employed in conventional immunohistological methods to immunostain tissue sections as described by, e.g., U.S. Pat. No. 6,017,949; Dellas et al., Gyn. Oncol. 1997, 67, 27-33; and others. Other markers for angiogenesis, include, e.g., Vezf1 (e.g., Xiang et al., Dev. Bio. 1999, 206, 123-141), angiopoietin, Tie-1, and Tie-2 (e.g., Sato et al., Nature 1995, 376, 70-74).

### Assays

Activity of the drug combinations of the present invention can be determined according to any effective in vitro or in vivo method.

### Raf/MEK/ERK Activity

A c-Raf kinase assay can be performed with a c-Raf enzyme activated (phosphorylated) by Lck kinase. Lck-activated c-Raf (Lck/c-Raf) is produced in Sf9 insect cells by co-infecting cells with baculoviruses expressing, under the control of the polyhedrin promoter, GST-c-Raf (from amino acid 302 to amino acid 648) and Lck (full-length). Both baculoviruses are used at the multiplicity of infection of 2.5 and the cells are harvested 48 hours post infection.

MEK-1 protein is produced in Sf9 insect cells by infecting cells with the baculovirus expressing GST-MEK-1 (full-length) fusion protein at the multiplicity of infection of 5 and harvesting the cells 48 hours post infection. Similar purification procedure is used for GST-c-Raf 302-648 and GST-MEK-1.

Transfected cells are suspended at 100 mg of wet cell biomass per mL in a buffer containing 10 mM sodium phosphate, 140 mM sodium chloride pH 7.3, 0.5% Triton X-100 and the protease inhibitor cocktail. The cells are disrupted with a Polytron homogenizer and centrifuged 30,000 g for 30 minutes. The 30,000 g supernatant is applied onto GSH-Sepharose. The resin is washed with a buffer containing 50 mM Tris, pH 8.0, 150 mM NaCl and 0.01% Triton X-100. The GST-tagged proteins are eluted with a solution containing 100 mM Glutathione, 50 mM Tris, pH 8.0, 150 mM NaCl and 0.01% Triton X-100. The purified proteins are dialyzed into a buffer containing 20 mM Tris, pH 7.5, 150 mM NaCl and 20% Glycerol.

Test compounds are serially diluted in DMSO using three-fold dilutions to stock concentrations ranging typically from 50 μM to 20 nM (e.g., final concentrations in the assay can range from 1 μM to 0.4 nM). The c-Raf biochemical assay is performed as a radioactive filtermat assay in 96-well Costar polypropylene plates (Costar 3365). The plates are loaded with 75 μL solution containing 50 mM HEPES pH 7.5, 70 mM NaCl, 80 ng of Lck/c-Raf and 1 μg MEK-1. Subsequently, 2 μL of the serially diluted individual compounds is added to the reaction, prior to the addition of ATP. The reaction is initiated with 25 μL ATP solution containing 5 μM ATP and 0.3 μCi [33P]-ATP. The plates were sealed and incubated at 32° C. for 1 hour. The reaction is quenched with the addition of 50 μl of 4% Phosphoric Acid and harvested onto P30 filtermats (PerkinElmer) using a Wallac Tomtec Harvester. Filtermats are washed with 1% Phosphoric Acid first and deinonized H₂O second. The filters are dried in a microwave, soaked in scintillation fluid and read in a Wallac 1205 Betaplate Counter (Wallac Inc., Atlanta, Ga., U.S.A.). The results are expressed as percent inhibition.

% Inhibition=[100−(Tib/Ti)]×100 where
Tib=(counts per minute with inhibitor)−(background)
Ti=(counts per minute without inhibitor)−(background)

Raf activity can also be monitored by its ability to initiate the cascade leading to ERK phosphorylation (i.e., raf/MEK/ERK), resulting in phospho-ERK. A Bio-Plex Phospho-ERK1/2 immunoassay can be performed as follows:

A 96-well phospho-ERK (pERK) immunoassay, using laser flow cytometry platform has been established to measure inhibition of basal pERK in cell lines. MDA-MB-231 cells are plated at 50,000 cells per well in 96-well microtitre plates in complete growth media. For effects of test compounds on basal pERK1/2 inhibition, the next day after plating, MDA-MB-231 cells are transferred to DMEM with 0.1% BSA and incubated with test compounds diluted 1:3 to a final concentration of 3 mM to 12 nM in 0.1% DMSO. Cells are incubated with test compounds for 2 h, washed, and lysed in Bio-Plex whole cell lysis buffer A. Samples are diluted with buffer B 1:1 (v/v) and directly transferred to assay plate or frozen at −80 C degrees until processed. 50 mL of diluted MDA-MB-231 cell lysates are incubated with about 2000 of 5 micron Bio-Plex beads conjugated with an anti-ERK1/2 antibody overnight on a shaker at room temperature. The next day, biotinylated phospho-ERK1/2 sandwich immunoassay is performed, beads are washed 3 times during each incubation and then 50 mL of PE-strepavidin is used as a developing reagent. The relative fluorescence units of pERK1/2 is detected by counting 25 beads with Bio-Plex flow cell (probe) at high sensitivity. The IC50 is calculated by taking untreated cells as maximum and no cells (beads only) as background.

### Cell Proliferation

An example of a cell proliferation assay is described in the Examples below. However, proliferation assays can be performed by any suitable method. For example, a breast carcinoma cell proliferation assay can be performed as follows. Other cell types can be substituted for the MDA-MB-231 cell line.

Human breast carcinoma cells (MDA MB-231, NCI) are cultured in standard growth medium (DMEM) supplemented with 10% heat-inactivated FBS at 37° C. in 5% CO₂ (vol/vol) in a humidified incubator. Cells are plated at a density of 3000 cells per well in 90 μL growth medium in a 96 well culture dish. In order to determine T₀ₕ CTG values, 24 hours after plating, 100 μL of CellTiter-Glo Luminescent Reagent (Promega) is added to each well and incubated at room temperature for 30 minutes. Luminescence is recorded on a Wallac Victor II instrument. The CellTiter-Glo reagent results in cell lysis and generation of a luminescent signal proportional to the amount of ATP present, which, in turn is directly proportional to the number of cells present.

Test compounds are dissolved in 100% DMSO to prepare 10 mM stocks. Stocks are further diluted 1:400 in growth medium to yield working stocks of 25 μM test compound in 0.25% DMSO. Test compounds are serially diluted in growth medium containing 0.25% DMSO to maintain constant DMSO concentrations for all wells. 60 μL of diluted test compound are added to each culture well to give a final volume of 180 μL. The cells with and without individual test compounds are incubated for 72 hours at which time ATP dependent luminescence was measured, as described previously, to yield T₇₂ₕ values. Optionally, the IC₅₀ values can be determined with a least squares analysis program using compound concentration versus percent inhibition.

% Inhibition=[1−(T_{72h test}−T₀ₕ)/(T_{72h ctrl}−T₀ₕ)]×100, where
T_{72h test}=ATP dependent luminescence at 72 hours in the presence of test compound
T_{72h ctrl}=ATP dependent luminescence at 72 hours in the absence of test compound
T₀ₕ=ATP dependent luminescence at Time Zero.

### Angiogenesis

One useful model to study angiogenesis is based on the observation that, when a reconstituted basement membrane matrix, such as Matrigel, supplemented with growth factor (e.g., FGF-1), is injected subcutaneously into a host animal, endothelial cells are recruited into the matrix, forming new blood vessels over a period of several days. See, e.g., Passaniti et al., Lab. Invest., 67:519-528, 1992. By sampling the extract at different times, angiogenesis can be temporally dissected, permitting the identification of genes involved in all stages of angiogenesis, including, e.g., migration of endothelial cells into the matrix, commitment of endothelial cells to angiogenesis pathway, cell elongation and formation of sac-like spaces, and establishment of functional capillaries comprising connected, and linear structures containing red blood cells. To stabilize the growth factor and/or slow its release from the matrix, the growth factor can be bound to heparin or another stabilizing agent. The matrix can also be periodically re-infused with growth factor to enhance and extend the angiogenic process.

Other useful systems for studying angiogenesis, include, e.g., neovascularization of tumor explants (e.g., U.S. Pat. Nos. 5,192,744; 6,024,688), chicken chorioallantoic membrane (CAM) assay (e.g., Taylor and Folkman, Nature, 297:307-312, 1982; Eliceiri et al., J. Cell Biol., 140, 1255-1263, 1998), bovine capillary endothelial (BCE) cell assay (e.g., U.S. Pat. No. 6,024,688; Polverini, P. J. et al., Methods Enzymol., 198: 440-450, 1991), migration assays, HUVEC (human umbilical cord vascular endothelial cell) growth inhibition assay (e.g., U.S. Pat. No. 6,060,449).

### Additional Compounds Included in the Drug Combination of the Present Invention

The drug combinations of this invention can optionally be administered with one or more additional pharmaceutical agents where the combination causes no unacceptable adverse effects. This may be of particular relevance for the treatment of hyper-proliferative diseases such as cancer. In this instance, the drug combination of this invention can be combined with other known cytotoxic agents, signal transduction inhibitors, or with other anti-cancer agents, as well as with admixtures and combinations thereof.

In one embodiment, the drug combination of the present invention is used with additional cytotoxic anti-cancer agents. Examples of such agents can be found in the 11^{th} Edition of the Merck Index (1996). These agents include, by no way of limitation, asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other cytotoxic drugs suitable for use with the drug combination of the invention include, but are not limited to, those compounds acknowledged to be used in the treatment of neoplastic diseases in Goodman and Gilman's The Pharmacological Basis of Therapeutics (Ninth Edition, 1996, McGraw-Hill). These agents include, by no way of limitation, aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other cytotoxic anti-cancer agents suitable for use in combination with the drug combinations of the invention also include newly discovered cytotoxic principles such as oxaliplatin, gemcitabine, capecitabine, epothilone and its natural or synthetic derivatives, temozolomide (Quinn et al., J. Clin. Oncology 2003, 21(4), 646-651), tositumomab (Bexxar), trabedectin (Vidal et al., Proceedings of the American Society for Clinical Oncology 2004, 23, abstract 3181), and the inhibitors of the kinesin spindle protein Eg5 (Wood et al., Curr. Opin. Pharmacol. 2001, 1, 370-377).

In another embodiment, the drug combination of the present invention can be combined with other signal transduction inhibitors. Of particular interest are signal transduction inhibitors which target the EGFR family, such as EGFR, HER-2, and HER-4 (Raymond et al., Drugs 2000, 60 (Supp1.1), 15-23; Harari et al., Oncogene 2000, 19 (53), 6102-6114), and their respective ligands. Examples of such agents include, by no way of limitation, antibody therapies such as Herceptin (trastuzumab), Erbitux (cetuximab), and pertuzumab. Examples of such therapies also include, by no way of limitation, small-molecule kinase inhibitors such as ZD-1839/Iressa (Baselga et al., Drugs 2000, 60 (Suppl. 1), 33-40), OSI-774/Tarceva (Pollack et al. J. Pharm. Exp. Ther. 1999, 291(2), 739-748), CI-1033 (Bridges, Curr. Med. Chem. 1999, 6, 825-843), GW-2016 (Lackey et al., 92^{nd} AACR Meeting, New Orleans, Mar. 24-28, 2001, abstract 4582), CP-724,714 (Jani et al., Proceedings of the American Society for Clinical Oncology 2004, 23, abstract 3122), HKI-272 (Rabindran et al., Cancer Res. 2004, 64, 3958-3965), and EKB-569 (Greenberger et al., 11^{th} NCI-EORTC-AACR Symposium on New Drugs in Cancer Therapy, Amsterdam, Nov. 7-10, 2000, abstract 388).

In another embodiment, the drug combination of the present invention can be combined with other signal transduction inhibitors targeting receptor kinases of the split-kinase domain families (VEGFR, FGFR, PDGFR, flt-3, c-kit, c-fms, and the like), and their respective ligands. These agents include, by no way of limitation, antibodies such as Avastin (bevacizumab). These agents also include, by no way of limitation, small-molecule inhibitors such as STI-571/Gleevec (Zvelebil, Curr. Opin. Oncol., Endocr. Metab. Invest. Drugs 2000, 2(1), 74-82), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92^{nd} AACR Meeting, New Orleans, Mar. 24-28, 2001, abstract 3152), AG-13736 (Herbst et al., Clin. Cancer Res. 2003, 9, 16 (suppl 1), abstract C253), KRN-951 (Taguchi et al., 95^{th} AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539).

In another embodiment, the drug combinations of the present invention are used with inhibitors of the Raf/MEK/ERK transduction pathway (Avruch et al., Recent Prog. Horm. Res. 2001, 56, 127-155), or the PKB (akt) pathway (Lawlor et al., J. Cell Sci. 2001, 114, 2903-2910). These include, by no way of limitation, PD-325901 (Sebolt-Leopold et al., Proceedings of the American Association of Cancer Research 2004, 45, abstract 4003), and ARRY-142886 (Wallace et al., Proceedings of the American Association of Cancer Research 2004, 45, abstract 3891).

In another embodiment, the drug combinations of the present invention are used with inhibitors of histone deacetylase. Examples of such agents include, by no way of limitation, suberoylanilide hydroxamic acid (SAHA), LAQ-824 (Ottmann et al., Proceedings of the American Society for Clinical Oncology 2004, 23, abstract 3024), LBH-589 (Beck et al., Proceedings of the American Society for Clinical Oncology 2004, 23, abstract 3025), MS-275 (Ryan et al., Proceedings of the American Association of Cancer Research 2004, 45, abstract 2452), and FR-901228 (Piekarz et al., Proceedings of the American Society for Clinical Oncology 2004, 23, abstract 3028).

In another embodiment, the drug combinations of the present invention are used with other anti-cancer agents such as proteasome inhibitors, and m-TOR inhibitors. These include, by no way of limitation, bortezomib (Mackay et al., Proceedings of the American Society for Clinical Oncology 2004, 23, Abstract 3109), and CCI-779 (Wu et al., Proceedings of the American Association of Cancer Research 2004, 45, abstract 3849).

### EXAMPLES

Abbreviations used in this specification are as follows:

| | |
|---|---|
| HPLC | high pressure liquid chromatography |
| MS | mass spectrometry |
| ES | Electro spray |
| DMSO | Dimethylsulfoxide |
| MP | melting point |
| NMR | nuclear resonance spectroscopy |
| TLC | thin layer chromatography |
| Rt | room temperature |

### Preparation of Intermediates/Starting Materials for the Fluoro-Substituted Diaryl Ureas of Formula (I)

### 1) Preparation of 4-amino-3-fluorophenol

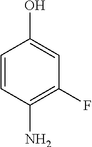

To a dry flask purged with Argon was added 10% Pd/C (80 mg) followed by 3-fluoro-4-nitrophenol (1.2 g, 7.64 mmol) as a solution in ethyl acetate (40 mL). The mixture was stirred under an H₂ atmosphere for 4 h. The mixture was filtered through a pad of Celite and the solvent was evaporated under reduced pressure to afford the desired product as a tan solid (940 mg, 7.39 mmol; 97% yield); ¹H-NMR (DMSO-d₆) 4.38 (s, 2H), 6.29-6.35 (m, 1H), 6.41 (dd, J=2.5, 12.7, 1H), 6.52-6.62 (m, 1H), 8.76 (s, 1H).

### 2) Preparation of 4-(4-amino-3-fluorophenoxy)pyridine-2-carboxylic acid methylamide

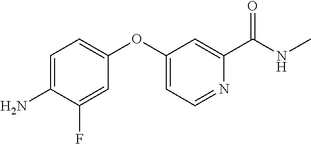 [SMILES: CNC(=O)c1cc(Oc2ccc(N)c(F)c2)ccn1]

A solution of 4-amino-3-fluorophenol (500 mg, 3.9 mmol) in N,N-dimethylacetamide (6 mL) cooled to 0° C. was treated with potassium tert-butoxide (441 mg, 3.9 mmol), and the brown solution was allowed to stir at 0° C. for 25 min. To the mixture was added 4-chloro-N-methyl-2-pyridinecarboxamide (516 mg, 3.0 mmol) as a solution in dimethylacetamide (4 mL). The reaction was heated at 100° C. for 16 h. The mixture was cooled to room temperature, quenched with H₂O (20 mL), and extracted with ehtylacetate (4×40 mL). The combined organics were washed with H₂O (2×30 mL), dried (MgSO₄), and evaporated to afford a red-brown oil. ¹H-NMR indicated the presence of residual dimethylacetamide, thus the oil was taken up in diethylether (50 mL) and was further washed with brine (5×30 mL). The organic layer was dried (MgSO₄) and concentrated to give 950 mg of the desired product as a red-brown solid, which was used in the next step without purification.

A method of preparing 4-chloro-N-methyl-2-pyridinecarboxamide is described in Bankston et al., Org. Proc. Res. Dev. 2002, 6(6), 777-781.

### Preparation of 4{4-[3-(4-chloro-3-trifluoromethylphenyl)-ureido]-3-fluorophenoxy}-pyridine-2-carboxylic acid methylamide, the Fluoro-Substituted Diaryl Urea of Formula (I)

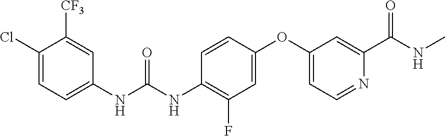 [SMILES: CNC(=O)c1cc(Oc2ccc(NC(=O)Nc3ccc(Cl)c(C(F)(F)F)c3)c(F)c2)ccn1]

To a solution of 4-(4-amino-3-fluorophenoxy)pyridine-2-carboxylic acid methylamide (177 mg, 0.68 mmol) in toluene (3 mL) was added 4-chloro-3-(trifluoromethyl)phenyl isocyanate (150 mg, 0.68 mmol). The mixture was stirred at rt for 72 h. The reaction was concentrated under reduced pressure and the residue was triturated with diethylether. The resulting solid was collected by filtration and dried in vacuo for 4 h to afford the title compound (155 mg, 0.32 mmol; 47% yield); ¹H-NMR (DMSO-d₆) 2.78 (d, J=4.9, 3H), 7.03-7.08 (m, 1H), 7.16 (dd, J=2.6, 5.6, 1H), 7.32 (dd, J=2.7, 11.6, 1H), 7.39 (d, J=2.5, 1H), 7.60 (s, 2H), 8.07-8.18 (m, 2H), 8.50 (d, J=5.7, 1H), 8.72 (s, 1H), 8.74-8.80 (m, 1H), 9.50 (s, 1H); MS (HPLC/ES) 483.06 m/z=(M+1).

### Preparation of a Solid Dispersions of 4{4-[3-(4-chloro-3-trifluoromethylphenyl)-ureido]-3-fluorophenoxy}-pyridine-2-carboxylic acid methylamide, the Fluoro-Substituted Diaryl Urea of Formula (I) with Polyvinylpyrrolidone

In an uncapped vial, one part of the compound 4}4-[3-(4-chloro-3-trifluoromethylphenyl)-ureido]-3-fluorophenoxy}-pyridine-2-carboxylic acid methylamide, the fluoro-substituted diaryl urea of Formula (I) as a free base was mixed with four parts polyvinylpyrrolidone (PVP-25/Kollidon® 25), and dissolved in a sufficient amount of a 1:1 mixture of acetone and ethanol, until all powders are in solution. The uncapped vial was placed into a vacuum oven set at 40° C., and let dry for at least 24-48 hours.

Alternatively, one part of the compound 4}4-[3-(4-chloro-3-trifluoromethylphenyl)-ureido]-3-fluorophenoxy}-pyridine-2-carboxylic acid methylamide of Formula (I) as a free base and three parts of polyvinylpyrrolidone (PVP 25/Kollidon® 25) are dissolved in 30 parts of a 80:20 acetone/ethanol mixture (w/w). Using a rotary vacuum evaporator the solvent was removed at 70° C. The dry residue was removed from the evaporation flask and sieved (630 μm).

In a further alternative embodiment, one part of the compound 4{4-[3-(4-chloro-3-trifluoromethylphenyl)-ureido]-3-fluorophenoxy}-pyridine-2-carboxylic acid methylamide of Formula (I) as a free base and seven parts PVP 25 are dissolved in 30 parts of a 80:20 acetone/ethanol mixture (w/w). Using a rotary vacuum evaporator the solvent was removed at 70° C. The dry residue was removed from the evaporation flask and sieved (630 μm).

### Solid Dispersion of 4{4-[3-(4-chloro-3-trifluoromethylphenyl)-ureido]-3-fluorophenoxyl-pyridine-2-carboxylic acid methyl amide if Formula (I) with PVP and Croscarmellose Sodium

A solution of 0.4 kg of the of the compound 4{4-[3-(4-chloro-3-trifluoromethylphenyl)-ureido]-3-fluorophenoxy}-pyridine-2-carboxylic acid methylamide of Formula I as a free base and 1.6 kg of PVP 25 in a mixture of 6.4 kg acetone and 1.6 kg ethanol was prepared. Using a fluidized bed vacuum granulator this solution was sprayed onto a powder bed of 2 kg croscarmellose sodium at a temperature of 60-70° C. After drying the product was sieved (1 mm). The granulate can be used as it is or it can be further formulated for example to sachet, capsule or tablet formulations. For example, the granulate was roller compacted and screened 3 and 1 mm. Subsequently the compacted granulate was blended with 0.54 kg croscarmellose sodium, 24 g colloidal anhydrous silica and 36 g magnesium stearate. This ready-to-press blend was compressed on a rotary tablet press to tablets containing 20, 50 an 100 mg of the compound of Formula I. The tablets may be film-coated for light protection.

### Preparation of 4{4-[3-(4-chloro-3-trifluoromethylphenyl)-ureido]-3-fluorophenoxy}-pyridine-2-carboxylic acid methylamide hydrochloride

The compound of example 1 as a free base (2.0 g) was dissolved in anhydrous tetrahydrofuran (15 mL) and a 4M HCl/dioxane was added (excess). The solution was then concentrated in vacuo to afford 2.32 grams of off-white solids. The crude salt was dissolved in hot ethanol (125 mL), activated carbon was added and the mixture heated at reflux for 15 minutes. The hot suspension was filtered through a pad of Celite 521 and allowed to cool to room temperature. The flask was placed in a freezer overnight. The crystalline solids were collected by suction filtration, washed with ethanol, then hexane and air-dried. The mother liquors were concentrated down and crystallization (in freezer) allowed taking place overnight. A second crop of solids was collected and combined with the first crop. The colorless salt was dried in a vacuum oven at 60° C. over two days. Yield of hydrochloride salt obtained 1.72 g (79%).

Melting point: 215° C.

Elemental analysis:

| | Calcd. | Found |
|---|---|---|
| C | 48.57 | 48.68 |
| H | 3.11 | 2.76 |
| N | 10.79 | 10.60 |
| Cl | 13.65 | 13.63 |
| F | 14.63 | 14.88 |

### Preparation of 4{4-[3-(4-chloro-3-trifluoromethylphenyl)-ureido]-3-fluorophenoxy}-pyridine-2-carboxylic acid methylamide mesylate

The compound of example 1 as a free base (2.25 g) was dissolved in ethanol (100 mL) and a stock solution of methanesulfonic acid (excess) was added. The solution was then concentrated in vacuo to afford a yellow oil. Ethanol was added and concentration repeated, affording 2.41 g of off-white solids. The crude salt was dissolved in hot ethanol (˜125 mL) and then cooled slowly to crystallize. After reaching room temperature, the flask was placed in a freezer overnight. The colorless crystalline material was collected by suction filtration; the filter cake was washed with ethanol, then hexane and air-dried, to afford 2.05 g of material, which was dried in a vacuum oven at 60° C. overnight.

Melting point: 231° C.

Elemental analysis:

| | Calcd. | Found |
|---|---|---|
| C | 45.64 | 45.34 |
| H | 3.31 | 3.08 |
| N | 9.68 | 9.44 |
| Cl | 6.12 | 6.08 |
| F | 13.13 | 13.42 |
| S | 5.54 | 5.59 |

### Preparation of 4{4-[3-(4-chloro-3-trifluoromethylphenyl)-ureido]-3-fluorophenoxy}-pyridine-2-carboxylic acid methylamide phenylsulfonate

The compound of example 1 as a free base (2.25 g) was suspended in ethanol (50 mL) and benzensulfonic acid (0.737 g) in ethanol (50 mL) was added. The mixture was heated with vigorous stirring. All solid material dissolved to give a reddish solution. The solution was allowed to cool to room temperature and the flask scratched. Crystal formation was difficult to achieve, some seeds were found, added to solution and placed in freezer overnight. Grayish-tan solids had formed in the flask; the material was broken up & collected by suction filtration. The solids were washed with ethanol, then hexane and air-dried. Weighed product: 2.05 g, 69% yield.

Melting point: 213° C.

Elemental Analysis:

| | Calcd. | Found |
|---|---|---|
| C | 50.59 | 50.24 |
| H | 3.30 | 3.50 |
| N | 8.74 | 8.54 |
| F | 11.86 | 11.79 |
| Cl | 5.53 | 5.63 |
| S | 5.00 | 5.16 |

c-raf (raf-1) Biochemical Assay

The c-raf biochemical assay was performed with a c-raf enzyme that was activated (phosphorylated) by Lck kinase. Lck-activated c-raf (Lck/c-raf) was produced in Sf9 insect cells by co-infecting cells with baculoviruses expressing, under the control of the polyhedrin promoter, GST-c-raf (from amino acid 302 to amino acid 648) and Lck (full-length). Both baculoviruses were used at the multiplicity of infection of 2.5 and the cells were harvested 48 h post infection.

MEK-1 protein was produced in Sf9 insect cells by infecting cells with the baculovirus expressing GST-MEK-1 (full-length) fusion protein at the multiplicity of infection of 5 and harvesting the cells 48 hours post infection. Similar purification procedure was used for GST-c-raf 302-648 and GST-MEK-1.

Transfected cells were suspended at 100 mg of wet cell biomass per mL in a buffer containing 10 mM sodium phosphate, 140 mM sodium chloride pH 7.3, 0.5% Triton X-100 and the protease inhibitor cocktail. The cells were disrupted with Polytron homogenizer and centrifuged 30,000 g for 30 minutes. The 30,000 g supernatant was applied onto GSH-Sepharose. The resin was washed with a buffer containing 50 mM Tris, pH 8.0, 150 mM NaCl and 0.01% Triton X-100. The GST-tagged proteins were eluted with a solution containing 100 mM Glutathione, 50 mM Tris, pH 8.0, 150 mM NaCl and 0.01% Triton X-100. The purified proteins were dialyzed into a buffer containing 20 mM Tris, pH 7.5, 150 mM NaCl and 20% Glycerol.

Test compounds were serially diluted in DMSO using three-fold dilutions to stock concentrations ranging typically from 50 μM to 20 nM (final concentrations in the assay range from 1 μM to 0.4 nM). The c-Raf biochemical assay was performed as a radioactive filtermat assay in 96-well Costar polypropylene plates (Costar 3365). The plates were loaded with 75 μL solution containing 50 mM HEPES pH 7.5, 70 mM NaCl, 80 ng of Lck/c-raf and 1 μg MEK-1. Subsequently, 2 μL of the serially diluted individual compounds were added to the reaction, prior to the addition of ATP. The reaction was initiated with 25 μL ATP solution containing 5 μM ATP and 0.3 μCi [33P]-ATP. The plates were sealed and incubated at 32° C. for 1 h. The reaction was quenched with the addition of 50 μL of 4% Phosphoric Acid and harvested onto P30 filtermats (PerkinElmer) using a Wallac Tomtec Harvester. Filtermats were washed with 1% Phosphoric Acid first and deinonized H₂O second. The filters were dried in a microwave, soaked in scintillation fluid and read in a Wallac 1205 Betaplate Counter (Wallac Inc., Atlanta, Ga., U.S.A.). The results were expressed as percent inhibition.

% Inhibition=[100−(T_{ib}/Tᵢ)]×100 where
T_{ib}=(counts per minute with inhibitor)−(background)
Tᵢ=(counts per minute without inhibitor)−(background)
   The fluoro-substituted diaryl urea of Formula (I) shows potent inhibition of raf kinase in this assay.

### p38 Kinase In Vitro Assay

Purified and His-tagged p38 α2 (expressed in E. Coli) was activated in vitro by MMK-6 to a high specific activity. Using a microtiter format, all reactions were conducted in 100 μL volumes with reagents diluted to yield 0.05 μg/well of activated p38 α2 and 10 μg/well of myelin basic protein in assay buffer (25 mM HEPES 7.4, 20 mM MgCl₂, 150 mM NaCl). Test compounds (5 μL of a 10% DMSO solution in water) were prepared and diluted into the assay to cover a final concentration range from 5 nM to 2.5 μM. The kinase assay was initiated by addition of 25 μL of an ATP cocktail to give a final concentration of 10 μM cold ATP and 0.2 μCi [gamma-³³P] ATP per well (200-400 dpm/pmol of ATP). The plate was incubated at 32° C. for 35 min., and the reaction quenched with 7 μL of a 1 N aq HCl solution. The samples were harvested onto a P30 Filtermat (Wallac, Inc.) using a TomTec 1295 Harvester (Wallac, Inc.), and counted in a LKB 1205 Betaplate Liquid Scintillation Counter (Wallac, Inc.). Negative controls included substrate plus ATP alone. SW1353 cellular assay: SW1353 cells (human chondro-sarcoma) are seeded (1000 cells/100 μL DMEM 10% FCS/well) into 96-well plates and incubated overnight. After medium replacement, cells are exposed to test compounds for 1 h at 37° C., at which time human IL-1 (1 ng/mL, Endogen, Woburn, Wash.) and recombinant human TNFalpha (10 ng/mL) are added. Cultures are incubated for 48 h at 37° C., then supernatant IL-6 values are determined by ELISA. The fluoro-substituted diaryl urea of Formula (I) shows significant inhibition of p38 kinase.

Bio-Plex Phospho-ERK ½ Immunoassay

A 96 well pERK immunoassay, using laser flow cytometry (Bio-Rad) platform has been established to measure inhibition of basal pERK in breast cancer cell line. MDA-MB-231 cells were plated at 50,000 cells per well in 96 well microtitre plates in complete growth media. For effects of test compounds on basal pERK1/2 inhibition, the next day after plating, MDA-MB-231 cells were transferred to DMEM with 0.1% BSA and incubated with test compounds diluted 1:3 to a final concentration of 3 μM to 12 nM in 0.1% DMSO. Cells were incubated with test compounds for 2 h, washed, and lysed in Bio-Plex whole cell lysis buffer A. Samples are diluted with buffer B 1:1 (v/v) and directly transferred to assay plate or frozen at −80 C. degrees until processed. 50 μL of diluted MDA-MB-231 cell lysates were incubated with about 2000 of 5 micron Bio-Plex beads conjugated with an anti-ERK1/2 antibody overnight on a shaker at room temperature. The next day, biotinylated phospho-ERK1/2 sandwich immunoassay was performed, beads are washed 3 times during each incubation and then 50 μL of PE-strepavidin was used as a developing reagent. The relative fluorescence units of pERK1/2 were detected by counting 25 beads with Bio-Plex flow cell (probe) at high sensitivity. The IC50 was calculated by taking untreated cells as maximum and no cells (beads only) as background using in an Excel spreadsheet based program.

The fluoro-substituted diaryl urea of Formula (I) shows significant inhibition in this assay.

Flk-1 (murine VEGFR-2) Biochemical Assay

This assay was performed in 96-well opaque plates (Costar 3915) in the TR-FRET format. Reaction conditions are as follows: 10 μM ATP, 25 nM poly GT-biotin, 2 nM Eu-labelled phospho-Tyr Ab, 10 nM APC, 7 nM Flk-1 (kinase domain), 1% DMSO, 50 mM HEPES pH 7.5, 10 mM MgCl₂, 0.1 mM EDTA, 0.015% BRIJ, 0.1 mg/mL BSA, 0.1% mercapto-ethanol). Reaction is initiated upon addition of enzyme. Final reaction volume in each well is 100 μL. Plates are read at both 615 and 665 nM on a Perkin Elmer Victor V Multilabel counter at about 1.5-2.0 hours after reaction initiation. Signal is calculated as a ratio: (665 nm/615 nm)*10000 for each well.

The fluoro-substituted diaryl urea of Formula (I) shows significant inhibition of VEGFR2 kinase.

### Murine PDGFR FRET Biochemical Assay

This assay was formatted in a 96-well black plate (Costar 3915). The following reagents are used: Europium-labeled anti-phosphotyrosine antibody pY20 (Perand streptavidin-APC; poly GT-biotin from, and mouse PDGFR. The reaction conditions are as follows: 1 nM mouse PDGFR is combined with 20 μM ATP, 7 nM poly GT-biotin, 1 nM pY20 antibody, 5 nM streptavidin-APC, and 1% DMSO in assay buffer (50 mM HEPES pH 7.5, 10 mM MgCl₂, 0.1 mM EDTA, 0.015% BRIJ 35, 0.1 mg/mL BSA, 0.1% mercaptoethanol). Reaction is initiated upon addition of enzyme. Final reaction volume in each well is 100 μL. After 90 minutes, the reaction is stopped by addition of 10 μL/well of 5 μM staurosporine. Plates are read at both 615 and 665 nm on a Perkin Elmer VictorV Multilabel counter at about 1 hour after the reaction is stopped. Signal is calculated as a ratio: (665 nm/615 nm)*10000 for each well.

The fluoro-substituted diaryl urea of Formula (I) shows significant inhibition of PDGFR kinase.

For IC₅₀ generation for both PDGFR and Flk-1, compounds were added prior to the enzyme initiation. A 50-fold stock plate was made with compounds serially diluted 1:3 in a 50% DMSO/50% dH2O solution. A 2 μL addition of the stock to the assay gave final compound concentrations ranging from 10 μM-4.56 nM in 1% DMSO. The data were expressed as percent inhibition: % inhibition=100−((Signal with inhibitor-background)/(Signal without inhibitor-background))*100

### MDA-MB231 Proliferation Assay

Human breast carcinoma cells (MDA MB-231, NCI) were cultured in standard growth medium (DMEM) supplemented with 10% heat-inactivated FBS at 37° C. in 5% CO₂ (vol/vol) in a humidified incubator. Cells were plated at a density of 3000 cells per well in 90 μL growth medium in a 96 well culture dish. In order to determine T₀ₕ CTG values, 24 hours after plating, 100 μL of CellTiter-Glo Luminescent Reagent (Promega) was added to each well and incubated at room temperature for 30 minutes. Luminescence was recorded on a Wallac Victor II instrument. The CellTiter-Glo reagent results in cell lysis and generation of a luminescent signal proportional to the amount of ATP present, which, in turn is directly proportional to the number of cells present.

Test compounds are dissolved in 100% DMSO to prepare 10 mM stocks. Stocks were further diluted 1:400 in growth medium to yield working stocks of 25 μM test compound in 0.25% DMSO. Test compounds were serially diluted in growth medium containing 0.25% DMSO to maintain constant DMSO concentrations for all wells. 60 μL of diluted test compound were added to each culture well to give a final volume of 180 μL. The cells with and without individual test compounds were incubated for 72 hours at which time ATP dependent luminescence was measured, as described previously, to yield T₇₂ₕ values. Optionally, the IC₅₀ values can be determined with a least squares analysis program using compound concentration versus percent inhibition.

% Inhibition=[1−(T_{72h test}−T₀ₕ)/(T_{72h ctrl}−T₀ₕ)]×100, where
T_{72h test}=ATP dependent luminescence at 72 hours in the presence of test compound
T_{72h ctrl}=ATP dependent luminescence at 72 hours in the absence of test compound
T₀ₕ=ATP dependent luminescence at Time Zero

The fluoro-substituted diaryl urea of Formula (I) shows significant inhibition of proliferation using this assay.

pPDGFR-Beta Sandwich ELISA in AoSMC Cells

100K P3-P6 Aortic SMC were plated in each well of 12-well cluster in 1000 μL volume/well of SGM-2 using standard cell culture techniques. Next day, cells were rinsed with 1000 μL D-PBS once, then serum starved in 500 μL SBM (smooth muscle cell basal media) with 0.1% BSA overnight. Compounds were diluted at a dose range from (10 μM to 1 nM in 10-fold dilution steps in DMSO. Final DMSO concentration 0.1%). Remove old media by inversion into the sink quickly then add 100 μL of each dilution to corresponding well of cells for 1 h at 37° C. Cells were then stimulated with 10 ng/mL PDGF-BB ligand for 7 min at 37° C. The media is decanted and 150 μL of isotonic lysis buffer with protease inhibitor tablet (Complete; EDTA-free) and 0.2 mM Na vanadate is added. Cells are lysed for 15 min at 4° C. on shaker in cold room. Lysates are put in eppendorf tubes to which 15 μL of agarose-conjugated anti-PDGFR-beta antibody is added and incubated at 4° C. overnight. Next day, beads are rinsed in 50-volumes of PBS three times and boiled in 1×LDS sample buffer for 5 minutes. Samples were run on 3-8% gradient Tris-Acetate gels and transferred onto Nitrocellulose. Membranes were blocked in 1% BSA/TBS-T for 1 hr. before incubation in anti-phospho-PDGFR-b (Tyr-857) antibody in blocking buffer (1:1000 dilution) for 1 h. After three washes in TBS-T, membranes were incubated in Goat anti-rabbit HRP IgG (1:25000 dilution) for 1 hr. Three more washes followed before addition of ECL substrate. Membranes were exposed to Hyperfilm-ECL. Subsequently, membranes were stripped and reprobed with anti-PDGFR-beta antibody for total PDGFR-beta.

Table 1 illustrates the results of in vitro kinase biochemical assays for p38 kinase, PDGFR kinase and VEGFR2 kinase. These three kinase targets are all involved in stroma activation and endothelial cell proliferation, leading to angiogenesis, and providing blood supply to the tumor tissue.

TABLE 1

| | mPDGFR | mVEGFR2 | p38 |
|---|---|---|---|
| | IC50, nM | IC50, nM | IC50, nM |
| Example 1 | 83 | 5.5 | 24 |

Table 2 illustrates the results of two cellular assays for raf kinase activity, which are (i) inhibition of pERK in MDA-MB231 cells, a mechanistic readout of raf kinase activity, and (ii) a proliferation assay of MDA-MB231 cells, a functional assay of raf kinase activity. In addition, Table 2 illustrates the results of PDGFR driven phosphorylation of PDGFR-beta in aortic smooth muscle cells, which is a mechanistic readout of PDGFR kinase inhibition.

TABLE 2

| | pERK in cells | Proliferation | pPDGFR |
|---|---|---|---|
| | (MDA-MB-231) | (MDA-MB-231) | (AoSMC) |
| | IC50, nM | IC50, nM | IC50, nM |
| Example 1 | 22 | 600 | 43.6 |

### Metabolite Profiles in Animals

N-oxide (M-2), hydroxymethyl (M-3), de-methylated (M-4) and N-oxide-demethylated (M-5) metabolites of the fluoro-substituted diaryl urea of Formula (I) are shown below. They were identified as metabolites of regorafenib in vitro upon incubation with liver enzymes of various mammalian species (man, dog, rat, mouse). Studies reveal that its metabolites show high protein binding in man and animal species (data not shown).

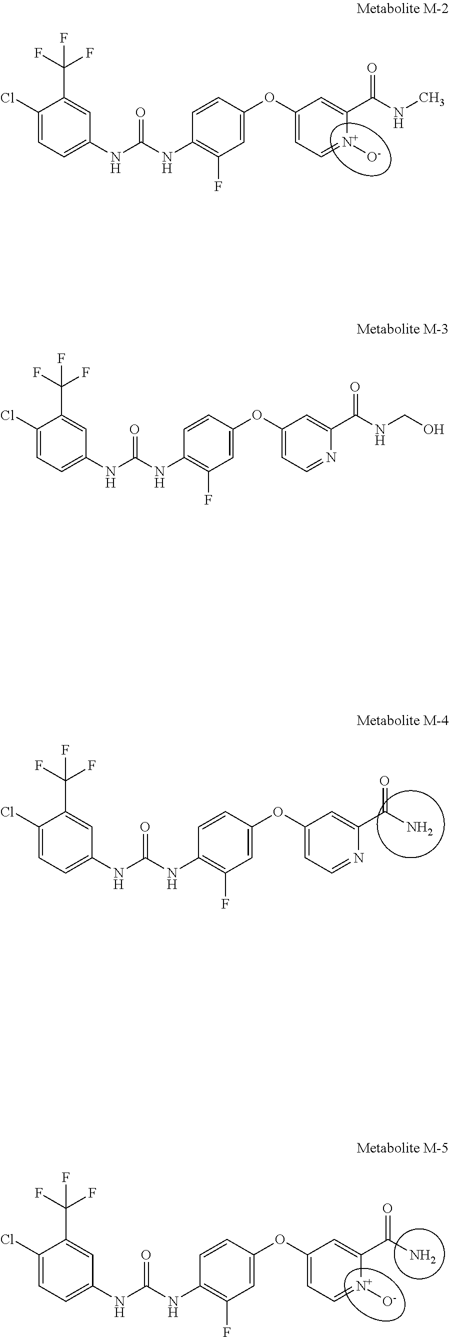 [SMILES: CNC(=O)c1cc(Oc2ccc(NC(=O)Nc3ccc(Cl)c(C(F)(F)F)c3)c(F)c2)cc[n+]1[O-] ; O=C(Nc1ccc(Cl)c(C(F)(F)F)c1)Nc1ccc(Oc2ccnc(C(=O)NCO)c2)cc1F ; NC(=O)c1cc(Oc2ccc(NC(=O)Nc3ccc(Cl)c(C(F)(F)F)c3)c(F)c2)ccn1 ; NC(=O)c1cc(Oc2ccc(NC(=O)Nc3ccc(Cl)c(C(F)(F)F)c3)c(F)c2)cc[n+]1[O-]]

The metabolite profiles are presented in Table 1, below.

TABLE 1
Metabolite profiles in incubations of [14C]
regorafenib(20 μM) with liver microsomes of different
species (protein concentration 0.5 mg/mL, 60 min).

| | % of radioactivity | | | |
|---|---|---|---|---|
| Metabolite | Man | Beagle dog | Wistar rat | NMRI mouse |
| M-2 | 29.7 | 3.6 | 5.4 | 26.9 |
| M-3 | 7.7 | 25.6 | 19 | 12 |
| Regorafenib | 59.1 | 70.8 | 75.7 | 57.8 |

The minor metabolite M-5 is not reported here because it could not be seperated chromatographically from a metabolite M-1 in this experiment

### Pharmacological Profiling

After oral administration of 10 mg/kg regorafenib to mice for 5 days, the N-oxide (M-2) exposure accounted for ˜16% of the total AUC (R+M-2+M-5), whereas the contribution of M-5 was ˜2% relative to total AUC. Data are shown in Table 2, below. Following oral administration of 10 mg/kg M-2 to mice, regorafenib exposure reached ˜17% of total exposure, indicating reduction of the N-oxide to be a relevant metabolic pathway in vivo, whereas M-5 accounted for 5% of total exposure.

TABLE 2
Pharmacokinetic parameters of regorafenib and its metabolites M-2 and
M-5 at steady state after their oral administration to female NMRI-Foxn-1
mice.

| Test | | | | Metabolite | | | Metabolite |
|---|---|---|---|---|---|---|---|
| compound | Regorafenib (R) | | | M-2 | | | M-5¹ |
| Analyte | R | M-2 | M-5 | R | M-2 | M-5 | M-5 |
| AUC | 38649 | 7490 | 772 | 11601 | 52101 | 3391 | 52796 |
| (μg · h/L) | | | | | | | |
| AUC | 82 | 16 | 2 | 17 | 78 | 5 | 100 |
| (% of | | | | | | | |
| total)² | | | | | | | |
| Cₘₐₓ | 4146 | 753 | 62.9 | 967 | 6150 | 342 | 5284 |
| (μg/L) | | | | | | | |

¹Only M-5 was measured;
²Differences in molecular weight were neglected Dose: 10 mg/kg; application: per os: qdx5; formulation: 1,2 propylene glycol/polyethylene glycol 400/pluronic F68 42.4/42.5/15 + 20% aqua [symbol] [symbol]
indicates data missing or illegible when filed

Synthetic metabolites dosed orally exhibited potent dose-dependent tumor growth inhibition (TGI) in preclinical murine HT-29 colorectal and MDA-MB-231 breast cancer xenografts, achieving significant TGI of 62/58% and 54/50%, respectively, compared with vehicle controls at 10 mg/kg.

### In Vivo Pharmacological Studies

In colon cancer patients treated daily with 160 mg coprecipitate tablets for a period of 21 days, M-2 and M-5 showed systemic exposures very similar to regorafenib. Results are presented in Table 4. As shown in Table 4, bio-transformation of regorafenib in patients with colorectal cancer results in significant elevation of demethylated and oxidized M-2 and M-5 synthetic metabolite levels. The synthetic metabolites were observed after single or multiple qd dosing with 160 mg co-precipitate tablets.

Continuous qd dosing in patients with colorectal cancer for 19 days resulted in a 25- and 2-fold increase in the AUC of M-5 and M-2 synthetic metabolite, respectively. Cmax values were similarly increased 42-fold and 5-fold, respectively. Values are reflected in comparison to the first dose.

In order to further characterize the metabolites, plasma concentrations of regorafenib, M-2 and M-5 were observed on a daily basis on day 1 and day 21 following administration of 160 mg Regorafenib co-precipitate tablet to patients with colorectal cancer (n=9, preliminary data). Combined Cmax level of the parent compound and M2/M5 metabolites was 11 mg/L on day 21. The combined pharmacologically relevant plasma concentration was evaluated to ˜2.5 mg/L, remaining 3 days after the last dosing.

TABLE 4
Pharmacokinetic parameters of regorafenib, M-2 and M-5 on day 1 and day
21, following administration of 160 mg regorafenib co-precipitate tablet to patients
with colorectal cancer (geometric mean (% CV), preliminary data)

| | Day 1 | | Day 21 | | | |
|---|---|---|---|---|---|---|
| | (n = 9) | | (n = 9) | | Fold | |
| | AUC | Cₘₐₓ | AUC [symbol] | C_{max,} [symbol] | change | |
| Compound | (mg · h/L) | (mg/L) | (mg · h/L) | (mg/L) | AUC | Cₘₐₓ |
| Regorafenib | 72.6 | 2.42 | 61.5 | 4.17 | 0.8 | 1.7 |
| | (47.6) | (36.9) | (44.0) | (44.7) | | |
| Metabolite M-2 | 24.0 | 0.764 | 55.4 | 3.57 | 2.3 | 4.7 |
| | (75.5) | (71.6) | (76.5) | (82.2) | | |
| Metabolite M-5 | 2.06 | 0.0755 | 51.8 | 3.14 | 25.1 | 41.6 |
| | (114)* | (107) | (101) | (103) | | |

*AUC [symbol] as t [symbol] could not be calculated [symbol]
indicates data missing or illegible when filed

### PROPHETIC EXAMPLES

### Procedure A

In this trial, Regorafenib is administered to screened patients in a sequential dosing with a seven day wash out period before the next infusion of Premetrexed and cisplatin. Regorafenib is administered at a dose of 160 mg qd from Day 2 to Day 14 followed by a 7 day break. Pharmokinetics of Regorafenib are assessed on Day 14 of cycle 1 and Day 1 of cycle 2.

### Procedure B

In this trial, Regorafenib is administered to screened patients continuously (160 mg qd) from Day to day 21. In cycle 1, Regorafenib dosing will begin on Day 2 in order to assess the pharmacokinetics of Pemetrexed and cisplatin without concomitant Regorafenib dosing. Pharmacokinetics of Regorafenib are assessed on Day 21 of cycle 1 and on Day 1 of cycle 2.

It is believed that one skilled in the art, using the preceding information and information available in the art, can utilize the present invention to its fullest extent. It should be apparent to one of ordinary skill in the art that changes and modifications can be made to this invention without departing from the spirit or scope of the invention as it is set forth herein.

The topic headings set forth above and below are meant as guidance where certain information can be found in the application, but are not intended to be the only source in the application where information on such topic can be found.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

The entire disclosure of all applications, patents and publications cited above and below, are incorporated in this application by reference in their entirety.

1. A combination which is therapeutically effective for the treatment of mammalian cancer comprising:
(1) a fluoro-substituted-diaryl urea of Formula (I), or a polymorph, solvate, hydrate, metabolite, prodrug, pharmaceutically acceptable salt or isolated diastereoisomer thereof, 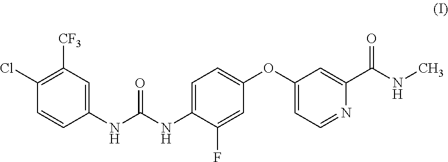 [SMILES: CNC(=O)c1cc(Oc2ccc(NC(=O)Nc3ccc(Cl)c(C(F)(F)F)c3)c(F)c2)ccn1]
and
(2) at least one antifolate.

2. A combination of claim 1 wherein the antifolate is
Trimethoprim (5-(3,4,5-trimethoxybenzyl)pyrimidine-2,4-diamine);
Pyrimethamine (5-(4-chlorophenyl)-6-ethyl-2,4-pyrimidinediamine);
a glutamic acid derivative of the formula X or a polymorph, solvate, hydrate, metabolite, prodrug, pharmaceutically acceptable salt or isolated diastereoisomer thereof; or
a glutamic acid derivative of the formula XX or a polymorph, solvate, hydrate, metabolite, prodrug, pharmaceutically acceptable salt or isolated diastereoisomer thereof: 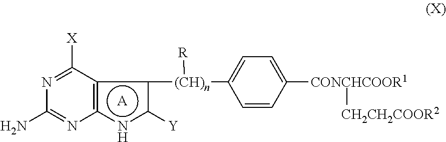
wherein
ring A is a pyrrole or pyrroline ring,
X is an amino group or a hydroxyl group,
Y is a hydrogen atom, an amino group or a hydroxyl group,
R is independently a hydrogen atom, a fluorine atom, a C₁₋₆ alkyl group, alkenyl group or alkynyl group,
R¹ and R² are independently a hydrogen atom or C₁₋₆ alkyl and n is an integer of 2 to 4; 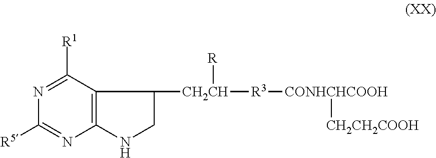
wherein R¹ is —OH or —NH₂,
R³ is 1,4-phenylene or 1,3-phenylene unsubstituted or substituted with chloro, fluoro, methyl, methoxy, or trifluoromethyl; thienediyl or furanediyl each unsubstituted or substituted with chloro, fluoro, methyl, methoxy, or
trifluoromethyl; cyclohexanediyl; or alkanediyl;
R is hydrogen, methyl, or hydroxymethyl; and
R^{5′} is hydrogen or alkyl of 1 to 6 carbon atoms.

3. A combination of claim 1 wherein the antifolate is Pemetrexed, (S)-2-[4-[2-(4-amino-2-oxo-3,5,7-triazabicyclo[4.3.0]nona-3,8,10-trien-9-yl)ethyl]benzoyl]aminopentanedioic acid or a polymorph, solvate, hydrate, metabolite, prodrug, pharmaceutically acceptable salt or an isolated diastereoisomer thereof.

4. A combination of claim 1 which additionally comprises (3) at least one platinum complex antineoplastic nucleic acid binding agent.

5. A combination of claim 4 wherein the platinum complex antineoplastic nucleic acid binding agent is
Cisplatin, (cis-diamminedichloroplatinum(II));
Carboplatin, (cis-diammine(cyclobutane-1,1-dicarboxylate-O,O′)platinum(II);
Oxaliplatin, ([(1R,2R)-cyclohexane-1,2-diamine](ethanedioato-O,O′)platinum(II));
Tetraplatin or Ormaplatin ((1R,2R)-cyclohexane-1,2-diamine platinum(IV) tetrachloride)
Satraplatin ((OC-6-43)bis(acetato)aminedichloro(cyclohexylamine)platinum), or a polymorph, solvate, hydrate, metabolite, prodrug, pharmaceutically acceptable salt or isolated diastereoisomer thereof.

6. A combination of claim 1 comprising:
(1) a fluoro-substituted-diaryl urea of Formula (I), or a metabolite thereof, or pharmaceutically acceptable salt thereof, 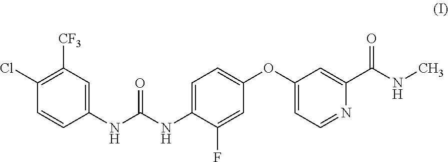 [SMILES: CNC(=O)c1cc(Oc2ccc(NC(=O)Nc3ccc(Cl)c(C(F)(F)F)c3)c(F)c2)ccn1]
(2) Pemetrexed, (S)-2-[4-[2-(4-amino-2-oxo-3,5,7-triazabicyclo[4.3.0]nona-3,8,10-trien-9-yl)ethyl]benzoyl]aminopentanedioic acid or a pharmaceutically acceptable salt thereof and
(3) Cisplatin, (cis-diamminedichloroplatinum(II)).

7. A combination of claim 1 adapted for administration of components (1) and (2) to a patient in need thereof either
(a) in the same formulation,
(b) in separate formulations using the same administration route, or
(c) in separate formulations using different administration routes.

8. A combination of claim 7 adapted for administration of components (1), (2) and (3) to a patient in need thereof either
(a) in the same formulation,
(b) in separate formulations using the same administration route, or
(c) in separate formulations using different administration routes.

9. A combination of claim 7 adapted for administration of components (1) and (2) to a patient in need thereof by oral delivery and/or by intravenous injection, infusion, intramuscular, subcutaneous or parenteral route of administration.

10. A combination of claim 8 adapted for administration of components (1), (2) and (3) to a patient in need thereof by oral delivery and/or by intravenous injection, infusion, intramuscular, subcutaneous or parenteral route of administration.

11. A combination of claim 7 adapted for concurrent administration of components (1) and (2) to a patient in need thereof.

12. A combination of claim 8 adapted for concurrent administration of components (1), (2) and (3) to a patient in need thereof.

13. A combination of claim 1 adapted for administration component (1) at a dosage within the range from about 0.1 to about 300 mg/kg of total body weight.

14. A combination of claim 13 adapted for administration component (2) as a solution 10-500 mg/m² of the patient surface area via injection.

15. A combination of claim 13 adapted for administration component (3) platinum complex as single dose of from 50 to 100 mg/m² (patient surface area) intended for a 3-4 week period or multiple doses of from 15 to 20 mg/m² over 5 days intended for a 3-4 week period.

16. A kit for treating mammalian cancer comprising:
(1) a fluoro-substituted-diaryl urea of Formula (I), or a polymorph, solvate, hydrate, metabolite, prodrug, pharmaceutically acceptable salt or isolated diastereoisomer thereof, 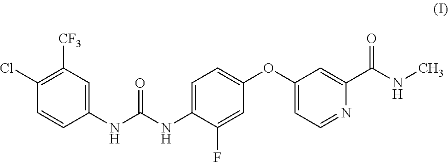 [SMILES: CNC(=O)c1cc(Oc2ccc(NC(=O)Nc3ccc(Cl)c(C(F)(F)F)c3)c(F)c2)ccn1]
and
(2) at least one antifolate.

17. A kit of claim 16 wherein the antifolate is Pemetrexed, (S)-2-[4-[2-(4-amino-2-oxo-3,5,7-triazabicyclo[4.3.0]nona-3,8,10-trien-9-yl)ethyl]benzoyl]aminopentanedioic acid or a pharmaceutically acceptable salt thereof.

18. A kit of claim 16 which additionally comprises
(3) at least one platinum complex antineoplastic nucleic acid binding agent.

19. A kit of claim 18 wherein the platinum complex antineoplastic nucleic acid binding agent is Cisplatin, (Platinol®) (cis diamminedichloroplatinum(II)).

20. A kit of claim 16 comprising separate doses of components (1) and (2), in separate containers.

21. A kit of claim 18 comprising separate doses of components (1), (2) and (3), in separate containers.

22. A kit of claim 21 for the treatment of non-small cell lung carcinoma.

23. A pharmaceutical composition for the treatment of mammalian cancer comprising a combination of claim 1 and at least one pharmaceutically acceptable carrier.

24. A pharmaceutical composition of claim 23 wherein the mammalian cancer to be treated is non-small cell lung cancer, cancer of the colon, pancreas, prostate, liver, kidney, lung, head and neck, pancreas, thyroid and ovaries.

25. A combination as in claim 1, wherein the mammalian cancer to be treated is non-small cell lung cancer and cancer of the colon, pancreas, prostate, liver, kidney, lung, head and neck, pancreas, thyroid and ovaries.

26. A pharmaceutical composition as in claim 23, wherein the mammilian cancer to be treated is
(a) small-cell lung cancer, non-small-cell lung cancer, bronchial adenoma, pleuropulmonary blastoma;
(b) brain stem and hypophtalmic glioma, cerebellar and cerebral astrocytoma, medulloblastoma, ependymoma, and neuroectodermal, pineal tumor;
(c) endometrial cancer, cervical cancer, ovarian cancer, vaginal cancer, vulvar cancer, sarcoma of the uterus bone;
(d) anal cancer, colon cancer, colorectal cancer, esophageal cancer, gallbladder cancer, gastric cancer, pancreatic cancer, rectal cancer, small intestine cancer, salivary gland cancer;
(e) hepatocellular carcinoma, cholangiocarcinoma, mixed hepatocellular cholangiocarcinoma;
(f) bladder cancer, penile cancer, kidney cancer, kidney neoplasm, renal pelvis cancer, ureter cancer, urethral cancer;
(g) squamous cell carcinoma, Kaposi's sarcoma, malignant melanoma, Merkel cell skin cancer, non-melanoma skin cancer;
(h) intraocular melanoma, retinoblastoma;
(i) laryngeal, hypopharyngeal, nasopharyngeal, oropharyngeal cancers, lip and oral cavity cancer;
(j) testicular cancer, prostate cancer;
(k) AIDS-related lymphoma, non-Hodgkin's lymphoma, cutaneous T-cell lymphoma, Hodgkin's disease, lymphoma of the central nervous system;
(l) glioblastoma, hematologic malignancies, Lhermitte-Duclose disease, malignant glioma, multiple myeloma, myeloid metaplasia, myeloplastic syndromes;
(m) sarcoma of the soft tissue, osteosarcoma, malignant fibrous histiocytoma, lymphosarcoma, rhabdomyosarcoma, leimyosarcoma, liposarcoma;
(n) acute myeloid leukemia, acute lymphoblastic leukemia, chronic lymphocytic leukemia, chronic myelogenous leukemia, and hairy cell leukemia
(o) thyroid cancer or
(p) gastrointestinal stromal tumors (G.I.S.T).

27. A method of treating a hyperproliferative disorder in a patient comprising administering to said patient an effective amount of a combination of claim 1.

28. A method of claim 27 wherein said hyperproliferative disorder is brain tumor, breast cancer, bone sarcoma, bronchial premalignancy, endometrial cancer, glioblastoma, hematologic malignancies, hepatocellular carcinoma, Hodgkin's disease, kidney neoplasms, leukemia, leimyosarcoma, liposarcoma, lymphoma, Lhermitte-Duclose disease, malignant glioma, melanoma, malignant melanoma, metastases, multiple myeloma, myeloid metaplasia, myeloplastic syndromes, non-small cell lung cancer, pancreatic cancer, prostate cancer, renal cell carcinoma, rhabdomyosarcoma, soft tissue sarcoma, thyroid cancer, gastrointestinal stromal tumors, squamous epithelial carcinoma of the skin or a combination thereof.

29. A method for treating or preventing a disease in a human and/or other mammal which is a disorder mediated by VEGFR-2, PDGFR, raf, p38, and/or VEGF, said method comprising administering to a human or other mammal a drug combination of claim 1.

30. A method for treating or preventing one or more of the following conditions in humans and/or other mammals:
tumor growth, retinopathy, ischemic retinal-vein occlusion, retinopathy of prematurity, age related macular degeneration; rheumatoid arthritis, psoriasis, a bullous disorder associated with subepidermal blister formation, including bullous pemphigoid, erythema multiforme, or dermatitis herpetiformis, rheumatoid arthritis, osteoarthritis, septic arthritis, tumor metastasis, periodontal disease, cornal ulceration, proteinuria and coronary thrombosis from atherosclerotic plaque, aneurismal aortic, birth control, dystrophobic epidermolysis bullosa, degenerative cartilage loss following traumatic joint injury, osteopenias mediated by MMP activity, tempero mandibular joint disease or demyelating disease of the nervous system,
said method comprising administering to a human or other mammal, a drug combination of claim 1.

31. A combination of claim 1 which comprises an additional anti-cancer agent selected from asparaginase, bleomycin, carmustine, chlorambucil, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, vindesine, aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine, gemcitabine, capecitabine, epothilone and its natural or synthetic derivatives, tositumomab, trabedectin, and temozolomide, trastuzumab, cetuximab, bevacizumab, pertuzumab, ZD-1839 (Iressa), OSI-774 (Tarceva), CI-1033, GW-2016, CP-724,714, HKI-272, EKB-569, STI-571 (Gleevec), PTK-787, SU-11248, ZD-6474, AG-13736, KRN-951, CP-547,632, CP-673,451, CHIR-258, MLN-518, AZD-2171, PD-325901, ARRY-142886, suberoylanilide hydroxamic acid (SAHA), LAQ-824, LBH-589, MS-275, FR-901228, bortezomib, and CCI-779.

32. A combination of claim 1 wherein the metabolites of the compound of formula (I) are:
4{4-[3-(4-chloro-3-trifluoromethylphenyl)-ureido]-3-fluorophenoxy}-pyridine-2-carboxylic acid amide,
4{4-[3-(4-chloro-3-trifluoromethylphenyl)-ureido]-3-fluorophenoxy}-1-hydroxy-pyridine-2-carboxylic acid methylamide, or
4{4-[3-(4-chloro-3-trifluoromethylphenyl)-ureido]-3-fluorophenoxy}-1-hydroxy-pyridine-2-carboxylic acid amide.
